SEQUENCE LISTING (64 sequences)

SEQ ID NO: 1 - protein, 19 aa, synthetic construct
GIVLLATYEKIISIYFIFF

SEQ ID NO: 2 - protein, 4 aa, synthetic construct
SHFV

SEQ ID NO: 3 - protein, 12 aa, synthetic construct
ITHLGVIAKNSL

SEQ ID NO: 4 - protein, 5 aa, synthetic construct
VGARN

SEQ ID NO: 5 - protein, 12 aa, synthetic construct
VASVVIVMLLYM

SEQ ID NO: 6 - protein, 12 aa, synthetic construct
KLNDARREESMG

SEQ ID NO: 7 - protein, 4 aa, synthetic construct
KRTT

SEQ ID NO: 8 - protein, 11 aa, synthetic construct
AIVRGLSFSLM

SEQ ID NO: 9 - protein, 12 aa, synthetic construct
LMSFVTFFAVME

SEQ ID NO: 10 - protein, 5 aa, synthetic construct
DTGNY

SEQ ID NO: 11 - protein, 12 aa, synthetic construct
VGARNGGGYSSY

SEQ ID NO: 12 - protein, 10 aa, synthetic construct
VGARNXYSSY

SEQ ID NO: 13 - protein, 16 aa, synthetic construct
VGGGAGRGNGGGYSSY

SEQ ID NO: 14 - protein, 14 aa, synthetic construct
VGGGAGRGNXYSSY

SEQ ID NO: 15 - protein, 11 aa, synthetic construct
SHFVGGGYSSY

SEQ ID NO: 16 - protein, 9 aa, synthetic construct
SHFVXYSSY

SEQ ID NO: 17 - protein, 19 aa, synthetic construct
VASVVIVMLLYMGGGYSSY

SEQ ID NO: 18 - protein, 17 aa, synthetic construct
VASVVIVMLLYMXYSSY

SEQ ID NO: 19 - protein, 19 aa, synthetic construct
ITHLGVIAKNSLGGGYSSY

SEQ ID NO: 20 - protein, 17 aa, synthetic construct
ITHLGVIAKNSLXYSSY

SEQ ID NO: 21 - protein, 26 aa, synthetic construct
GIVLLATYEKIISIYFIFFGGGYSSY

SEQ ID NO: 22 - protein, 24 aa, synthetic construct
GIVLLATYEKIISIYFIFFXYSSY

SEQ ID NO: 23 - protein, 19 aa, synthetic construct
KLNDARREESMGGGGYSSY

SEQ ID NO: 24 - protein, 17 aa, synthetic construct
KLNDARREESMGXYSSY

SEQ ID NO: 25 - protein, 11 aa, synthetic construct
KRTTGGGYSSY

SEQ ID NO: 26 - protein, 9 aa, synthetic construct
KRTTXYSSY

SEQ ID NO: 27 - protein, 18 aa, synthetic construct
AIVRGLSFSLMGGGYSSY

SEQ ID NO: 28 - protein, 16 aa, synthetic construct
AIVRGLSFSLMXYSSY

SEQ ID NO: 29 - protein, 19 aa, synthetic construct
LMSFVTFFAVMEGGGYSSY

SEQ ID NO: 30 - protein, 17 aa, synthetic construct
LMSFVTFFAVMEXYSSY

SEQ ID NO: 31 - protein, 12 aa, synthetic construct
DTGNYGGGYSSY

SEQ ID NO: 32 - protein, 10 aa, synthetic construct
DTGNYXYSSY

SEQ ID NO: 33 - protein, 9 aa, synthetic construct
VGGGAGRGN

SEQ ID NO: 34 - protein, 4 aa, synthetic construct
YSSY

SEQ ID NO: 35 - protein, 12 aa, synthetic construct
IMVTESSDYSSY

SEQ ID NO: 36 - protein, 12 aa, synthetic construct
IMVTESSDASSA

SEQ ID NO: 37 - protein, 12 aa, synthetic construct
IMVTESSDWSSW

SEQ ID NO: 38 - protein, 12 aa, synthetic construct
IMVTKSSRFSSF

SEQ ID NO: 39 - protein, 12 aa, synthetic construct
TQSTKSSRYSSY

SEQ ID NO: 40 - protein, 12 aa, synthetic construct
IMVTESSRYSSY

SEQ ID NO: 41 - protein, 12 aa, synthetic construct
IMVTASSAYDDY

SEQ ID NO: 42 - protein, 12 aa, synthetic construct
IMVTASSAYRDY

SEQ ID NO: 43 - protein, 12 aa, synthetic construct
IMVTASSAYRRY

SEQ ID NO: 44 - protein, 12 aa, synthetic construct
IMVTASSDYSSY

SEQ ID NO: 45 - protein, 13 aa, synthetic construct
HIMVTESSDYSSY

SEQ ID NO: 46 - protein, 13 aa, synthetic construct
WIMVTESSDYSSY

SEQ ID NO: 47 - protein, 14 aa, synthetic construct
VVIMVTESSDYSSY

SEQ ID NO: 48 - protein, 14 aa, synthetic construct
SSIMVTESSDYSSY

SEQ ID NO: 49 - protein, 12 aa, synthetic construct
LIATESSDYSSY

SEQ ID NO: 50 - protein, 12 aa, synthetic construct
AQTTESSDYSSY

SEQ ID NO: 51 - protein, 12 aa, synthetic construct
IMVTASSAYSSY

SEQ ID NO: 52 - protein, 12 aa, synthetic construct
IMVTESSDYSSY

SEQ ID NO: 53 - protein, 12 aa, synthetic construct
IMVTEPPDYSSY

SEQ ID NO: 54 - protein, 13 aa, synthetic construct
CIMVTESSDYSSY

SEQ ID NO: 55 - protein, 12 aa, synthetic construct
IMVTESSDYSSY

SEQ ID NO: 56 - protein, 12 aa, synthetic construct
  MOD_RES 9: note = 4-amino-phenylalanine
IMVTESSDXSSY

SEQ ID NO: 57 - protein, 12 aa, synthetic construct
  MOD_RES 9: note = 4-fluoro-phenylalanine
IMVTESSDXSSY

SEQ ID NO: 58 - protein, 12 aa, synthetic construct
  SITE 1..12: note = All D-amino acids
IMVTESSDYSSY

SEQ ID NO: 59 - skipped

SEQ ID NO: 60 - protein, 26 aa, synthetic construct
EPIHRSTLTALLSSIMVTESSDYSSY

SEQ ID NO: 61 - protein, 12 aa, synthetic construct
IMVTNSSNWSSW

SEQ ID NO: 62 - protein, 12 aa, synthetic construct
IMVTESSDFSSF

SEQ ID NO: 63 - protein, 12 aa, synthetic construct
TQSTESSDYSSY

SEQ ID NO: 64 - protein, 12 aa, synthetic construct
IMVTDSSAYSSY

(19) United States
(12) Patent Application Publication
(10) Pub. No.: US 2025/0327770 A1
(43) Pub. Date: Oct. 23, 2025

(54) Protein-Derived Peptide Multiplexes for Biomimetic Detection of VOC Profiles

(71) Applicant: University of Washington, Seattle, WA (US)

(72) Inventors: John Devin MACKENZIE, Seattle, WA (US); Siddharth Rath, Seattle, WA (US); Oliver Nakano-Baker, Seattle, WA (US); Hanson Kwok Fong, Seattle, WA (US); Shalabh Shuklah, Seattle, WA (US); Richard Lee, Seattle, WA (US); Sami Dogan, Seattle, WA (US); Mehmet Sarikaya, Seattle, WA (US); Tatum Grace Hennig, Seattle, WA (US)

(21) Appl. No.: 18/873,325

(22) PCT Filed: Jun. 9, 2023
(86) PCT No.: PCT/US2023/068242

§ 371 Date: Dec. 9, 2024

Related U.S. Application Data
(60) Provisional application No. 63/351,065, filed on Jun. 10, 2022.

(51) Int. Cl. G01N 27/414; G01N 33/543; G01N 33/569

(52) U.S. Cl. CPC G01N 27/4145; G01N 27/4141; G01N 27/4146; G01N 33/5438; G01N 33/56983

(57) ABSTRACT

The present disclosure provides peptides, chimeric molecular constructs, and compositions thereof, that can bind volatile organic compounds (VOCs) and be utilized to distinguish VOC profiles, e.g., indicative of disease, as well as related methods. A VOC-based gas sensing approach described in the present specification can be an effective screening tool, due, at least in part, to its speed, ease of use, sensitivity, specificity, and because it does not rely on binding to specific nucleic acid fragments or proteins to function.

### CROSS REFERENCE TO RELATED APPLICATIONS

This application claims the benefit of U.S. Provisional Patent Application No. 63/351,065, filed Jun. 10, 2022, which is incorporated by reference herein in its entirety.

### STATEMENT REGARDING FEDERALLY SPONSORED RESEARCH

This invention was made with government support under Grant No. 3U01HL152401-02S1, awarded by the National Institutes of Health (NIH). The government has certain rights in the invention.

### REFERENCE TO AN ELECTRONIC SEQUENCE LISTING

The contents of the electronic sequence listing (22-0936-WO_ST26_Sequence_Listing.xml; Size: 55,761 bytes; and Date of Creation: Jun. 9, 2023) is herein incorporated by reference in its entirety.

### BACKGROUND OF THE DISCLOSURE

In animals, the olfactory system detects and discriminates volatile organic compounds (VOCs) by binding to olfactory receptor proteins that trigger signals to the brain. The smell-sensing mechanism is sufficiently sensitive and selective to rapidly discriminate tiny variations in thousands of molecules at once, enabling animals to characterize their chemical environment, detect dangers, find mates, and assess food sources or toxins.

Human breath contains a rich mixture of VOCs, presenting distinct VOC fingerprints that can be affected by many factors, including stress and disease. Some animals can recognize disease chemical signatures; for instance, dogs have been trained to detect cancers and COVID-19. Thus, the variations in exhaled VOC profiles can be leveraged to detect and diagnose diseases.

However, conventional VOC sensors, such as breath alcohol testers, have significantly lower sensitivity and selectivity across a broad spectrum of compounds and, therefore, lack the ability to sensitively and specifically detect an ensemble of VOCs specific to a given disease. Advanced sensors, such as aptamer and CNT-based disease sensors, employ a single sensing element or parent protein to target an analyte, such as viral RNA, or an antigen, such as SARS-COV-2 spike protein. While such tests are commonly used for diagnoses, they are typically monospecific, not VOC-based, necessitate semi-invasive sampling of bodily fluids, and are vulnerable to falling sensitivity, e.g., as a virus mutates. Other potentially sensitive and selective tests also often suffer complications as was seen during the COVID-19 pandemic when PCR RNA and antigen tests routinely showed false negative results for days after infection and symptom onset.

Thus, there is a need for improved detection, identification and diagnosis of the state of living organisms, such as humans and non-human animals. A VOC-based gas sensing approach described in the present specification can be an effective screening tool, due, at least in part, to its speed, ease of use, sensitivity, specificity, and because it does not rely on binding to specific nucleic acid fragments or proteins to function.

### SUMMARY OF THE DISCLOSURE

The present disclosure provides peptides, chimeric molecular constructs, and compositions thereof, that can bind volatile organic components (VOCs), as well as related methods. In one aspect, a peptide is provided of a sequence that shares significant identity with the amino acid sequence of SEQ ID NO:1, SEQ ID NO:3, SEQ ID NO:5, SEQ ID NO:6, SEQ ID NO: 8, SEQ ID NO: 10, or SEQ ID NO:33, up to and including 100% identity.

In another aspect, a chimeric molecular construct is provided that includes one or more peptides of a sequence sharing significant identity with the amino acid sequence of SEQ ID NOS: 1-10 or SEQ ID NO:33, up to and including 100% identity, in which the peptide is fused to one or more functional domain and one or more linker sequence linking the peptide and the functional domain. In some embodiments, the functional domain is a surface binding domain.

In another aspect, the disclosure provides a composition that includes one or more peptides or chimeric molecular constructs of the disclosure. In some embodiments, the composition includes one or more peptides of a sequence that shares significant identity with the amino acid sequence of SEQ ID NOS: 11-22, up to and including 100% identity. In some embodiments, the composition further includes one or more peptides of a sequence that shares significant identity with the amino acid sequence of SEQ ID NOS: 23-32.

In certain embodiments of the composition, one or more peptides or chimeric molecular constructs are bound to a surface, non-covalently in some embodiments. In embodiments, the surface includes one or more of a carbonaceous surface, a carbon nanotube surface, single or multilayer graphene or graphitic carbon surface, a one-dimensional semiconducting element surface, a two-dimensional semiconductor surface, an oxide, II-VI, III-V or group IV bulk or thin film semiconductor surface, a semiconductor or semimetal surface, a dielectric or protective layer surface, and the like. In some embodiments, the surface includes a carbon nanotube field-effect transistors (CNT-FET), or a graphene field effect transistor (gFET).

In another aspect, the disclosure provides a method for detecting, prognosing, or monitoring treatment for COVID-19 infection. In embodiments, the method includes contacting a composition that includes one or more peptides or chimeric molecular constructs of the disclosure with a biological sample; detecting volatile organic compounds in the biological sample by their binding to the peptides or chimeric molecular constructs present in the composition; and comparing a signature of VOCs present in the biological sample with a VOC signature characteristic of COVID-19, in which a VOC signature present in the sample that matches a VOC signature characteristic of COVID-19 serves to detect COVID-19 infection, prognosis, or response to treatment in the biological sample.

In some embodiments of the method, the comparing steps include the use of a computational model trained to differentiate biological samples with and without the VOC signature characteristic of COVID-19.

In another aspect, the disclosure provides a method for designing a peptide multiplex probe for detection of a volatile organic compound signature in a biological or simulated biological sample, including providing a biological sample VOC signature to be detected and identifying a plurality of odorant-binding proteins (OBPs) capable of detecting VOCs in the biological sample VOC signature; and training a computational model, using the VOC signature to be detected and the plurality of OBPs capable of detecting VOCs in the biological sample VOC signature, to perform functions including (i) identifying a closely-matching OBP structure in an OBP database and identifying ligand-binding residues in each OBP in the plurality of OBPs; (ii) extracting the ligand-binding amino acid residues in each OBP and rearranging the order of extracted amino acid residues to mimic their arrangement in space in the OBP, generating candidate peptide multiplex probes; (iii) testing the candidate peptide multiplex probes against the biological sample VOC signature to be detected using a machine learning model trained to differentiate biological samples with and without the VOC signature to be detected; and (iv) selecting those candidate peptide multiplex probes that best differentiate biological samples with and without the VOC signature to be detected.

### BRIEF DESCRIPTION OF THE DRAWINGS

FIG. 1 shows a schematic representing the design of a biomimetic sensor (“electronic nose”). Odorant-binding proteins (OBPs) were selected to allow machine learning algorithms to detect a disease VOC fingerprint. Peptide probe sequences that mimic OBP-VOC binding slots are integrated into modular peptides that bind to both target molecules and to a surface of a molecular electronic biomimetic sensor device (e.g., CNT-FET), in this case low dimension n-conjugated carbon allotropes. Finally, device arrays functionalized with peptides sense the VOC indicators of disease.

FIG. 2A to FIG. 2F shows a schematic of the multiplex sensor design process. FIG. 2A shows how simulated N-plex OBP sensors were designed to sense VOC profiles from a virtual population. A multiplex set of hero OBPs was chosen for its ability to support a machine learning classification model that could identify healthy and diseased individuals. FIG. 2B shows that for each chosen OBP protein, a close homological match was identified in the PDB (i.e., hero OBP MmedCSP3 matched with MbraCSP2 in the PDB). FIG. 2C illustrates that the residues in contact with a bound ligand were identified in the PDB structure. FIG. 2D illustrates how contact residue sites were often spread across multiple separate parts of protein tertiary structure (e.g., alpha-helices). FIG. 2E shows the sequence order of these residues, while FIG. 2F illustrates rearrangement of the residues to traveling salesman-ordered positions to mimic residue positions in space. These amino acid positions in the hero OBP became the probe portion of one chimeric molecular construct.

FIG. 3 shows OBP-VOC binding affinities for the ten OBPs selected to detect SARS-COV-2 infection in the presence of six confounding respiratory disease signatures. The displayed VOCs have known binding affinity to one of the ten OBPs and are commonly observed in human breath; the compounds are sorted with VOCs having the highest OBP binding affinity (lowest K_{D}) at the top. VOCs that are disease markers but do not bind to one of the ten OBPs were omitted.

FIG. 4 shows graphs illustrating functional validation of the chimeric molecular constructs binding to target VOCs from simulated breath using QCM. The flow rate of the target VOCs for each peptide is 10× lower than the confounding controls. A similar or greater reduction in QCM resonant frequency indicates binding to the target.

FIG. 5 shows a graph of surface plasmon resonance (SPR) data showing specific absorption of MmedPep1-GrBP peptide onto thin film carbon.

FIG. 6A illustrates CNT-FET device fabrication, including a microfabrication process flow for FET sensors and solution deposition method for creating CNT network sensing channels. FIG. 6B illustrates photoresist channel banks atop the quadruplex sensor whereby each sensing region shows eight replicate FET sensors for a total of 32 on the chip. FIG. 6C shows representative transfer curves that illustrate average device characteristics of non-functionalized CNT FET sensors.

FIG. 7A to FIG. 7C show CNT-FET device response for ethyl butyrate sensing. FIG. 7A shows schematic illustrating VOC exposure experiments. FIG. 7B shows transfer curves from FETs treated with MmedPep1-GrBP and BhorPep1-GrBP. Inset shows the same data with a log scale, dashed lines for MmedPep1-GrBP, and solid lines for BhorPep1-GrBP responses. FIG. 7C shows source-drain current at gate voltage of −1 V for each exposure condition. MmedPdp1-GrBP was designed to bind ethyl butyrate and shows a robust signal response upon exposure. In contrast, BhorPep1-GrBP was designed to be sensitive to octanal, hexanal, nonanal, tridecane, and dodecane and respond weakly to ethyl butyrate exposure. Mean values are reported; error bars represent standard error. (N=2).

FIG. 8 shows a quartz crystal microbalance (QCM) experimental setup. Carrier N₂ gas is humidified by bubbling it through temperature-controlled water (control) or an aqueous VOC column (test gas) and then introduced to the QCM chamber where it flows past the sensor surface.

### DETAILED DESCRIPTION OF THE DISCLOSURE

Herein, peptides, chimeric molecular constructs, and compositions thereof, that can bind volatile organic components (VOCs) and be utilized to distinguish VOC profiles, e.g., indicative of disease, as well as related methods are described. Such peptides, chimeric molecular constructs, and compositions thereof, when bound to a sensor surface, were demonstrated to sensitively and selectively bind to target VOCs.

A number of terms are introduced below:

As used herein, the term “peptide” is used in its broadest sense to refer to a sequence of subunit D-amino acids, L-amino acids, or combinations thereof (also including glycine and any other non-chiral amino acid or derivative thereof) including canonical and non-canonical amino acids. The peptide may have any structure, including but not limited to alpha-helical and peptidomimetic structures such as (beta) β-peptides. The polypeptides described herein may be chemically synthesized or recombinantly expressed.

As used herein, a “conservative amino acid substitution” or “conservative substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the physico-chemical properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Particular conservative substitutions include, but are not limited to, Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

The term “peptide multiplex probe” means a combination of two or more peptides or chimeric molecular constructs identified to detect VOCs in a biological sample that comprise a VOC signature characteristic of a particular disease or disorder. Such peptides or chimeric molecular constructs can be incorporated onto a surface, e.g., a sensor surface, and into a diagnostic device for detection of a VOC signature characteristic or diagnostic for a disease or disorder.

As used herein, a “carbonaceous surface” is any surface that naturally comprises or is modified to comprise any organic material that contains a large amount of carbon content (>50%). In various non-limiting embodiments, the carbonaceous surface may comprise a graphene, graphite, amorphous carbon, carbon nanotube (CNT), carbon black, a carbon surface (including but not limited to a graphite or carbon nanotube), and a diamond-containing surface, each of which may have sp2, sp3 or a mixture of the two types of chemical bonding characteristics. The surface may comprise a single layer, multiple layers, or discrete portions on the surface of the carbonaceous compound(s).

The terms “statistically significant” or “significantly” refer to statistical evidence that there is a difference. It is defined as the probability of making a decision to reject the null hypothesis when the null hypothesis is actually true. The decision is often made using a p-value.

Recitation of ranges of values herein are merely intended to serve as a shorthand method of referring individually to each separate value falling within the range, unless otherwise indicated herein, and each separate value is incorporated into the specification as if it were individually recited. For example, if a concentration range is stated as 1% to 50% (or degrees, mass amounts, and the like), it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

As used herein, the singular forms “a”, “an” and “the” include plural referents unless the context clearly dictates otherwise.

The term “about” means plus or minus 10% of the recited measurement.

Mammalian and non-mammalian animal breath contains a mixture of VOCs that can be affected by factors such as stress and disease, and diseases can cause distinctive patterns of altered or elevated levels of VOCs, or “fingerprints”, often overlapping each other. Herein, the disclosure demonstrates a-priori design of many VOC probes that cooperatively target a specific, multi-VOC disease fingerprint, which designed peptides exhibit preferential binding to their intended VOC targets. For the exemplary disease of COVID-19, the disclosure shows that a unique machine learning model with a many-to-many sensing space (also as described herein) evolutionarily enhanced the diagnostic accuracy of the device, resulting in detection of molecular targets (e.g., VOCs) with five peptide-based probes (or channels), which was sufficient to identify COVID-19 with high accuracy. In the presence of confounding disease, ten channels were sufficient to de-convolute multiple overlapping disease signatures within the complex matrix of VOCs in human breath. Thus, the disclosure provides that a disease or disorder with a characteristic VOC fingerprint can be identified and, further, isolated or deconvoluted from VOC alterations due to a confounding disease or disorder.

### Peptides

In one aspect, a peptide that binds one or more volatile organic compounds (VOCs) is provided. In some embodiments, the peptide includes an amino acid sequence that is at least 50% identical to the amino acid sequence of SEQ ID NO:1 to SEQ ID NO:10, or SEQ ID NO:33. In other embodiments, the peptide has an amino acid sequence at least 50% identical to SEQ ID NO:1, SEQ ID NO:3, SEQ ID NO:5, SEQ ID NO:6, SEQ ID NO:8, SEQ ID NO: 10, or SEQ ID NO:33. In other embodiments, the peptide includes an amino acid sequence at least 55%, 60%, 65%, 70%, 75%, 80%, 85%, 90%, 95%, or 100% identical to the amino acid sequence of SEQ ID NO:1 to SEQ ID NO:10, or SEQ ID NO:33, while in other embodiments, the peptide includes an amino acid sequence that is at least 55%, 60%, 65%, 70%, 75%, 80%, 85%, 90%, 95%, or 100% identical to the amino acid sequence of SEQ ID NO: 1, SEQ ID NO:3, SEQ ID NO:5, SEQ ID NO:6, SEQ ID NO:8, SEQ ID NO:10, or SEQ ID NO: 33.

Substitutions to the reference amino acid sequences of SEQ ID NO:1-10, and SEQ ID NO:33, as well as to all reference amino acid sequences with assigned SEQ ID NOS described herein, may include, for example, any naturally occurring amino acids and variants thereof, non-naturally occurring amino acids, non-proteinogenic amino acids, and poly-N-substituted glycine residues. (i.e., peptoids). An amino acid substitution can be conservative or non-conservative and either is contemplated for each substitution unless otherwise specified. While described in greater detail above, briefly a conservative (amino acid) substitution means a given amino acid can be replaced by a residue having similar physiochemical characteristics, e.g., polar for polar, or aliphatic for aliphatic, and the like. A non-conservative substitution is, therefore, replacement of an amino acid with another having dissimilar physicochemical characteristics, e.g., acidic for basic, or basic for hydrophobic, and the like.

In some embodiments, the peptide of SEQ ID NO:1-10, and SEQ ID NO:33, as well as a peptide that includes an amino acid sequence at least 50%, 55%, 60%, 65%, 70%, 75%, 80%, 85%, 90%, or 95% identical to the amino acid sequence of SEQ ID NO:1 to SEQ ID NO: 10, or SEQ ID NO:33, includes one or more conservative amino acid substitutions. In certain embodiments, all substitutions to the peptide of SEQ ID NO:1-10, and SEQ ID NO:33, as well as a peptide that includes an amino acid sequence at least 50%, 55%, 60%, 65%, 70%, 75%, 80%, 85%, 90%, or 95% identical to the amino acid sequence of SEQ ID NO: 1 to SEQ ID NO: 10, or SEQ ID NO:33, are conservative amino acid substitutions.

### Chimeric Molecular Constructs

In another aspect, a chimeric molecular construct is provided including one or more peptides, fused to one or more functional domains and one or more linker sequence linking the peptide and the functional domain. In some embodiments, the one or more peptides of the chimeric molecular construct includes an amino acid sequence at least 50%, 55%, 60%, 65%, 70%, 75%, 80%, 85%, 90%, 95%, or 100% identical to the amino acid sequence selected from SEQ ID NOS: 1-10 or SEQ ID NO:33 fused to one or more functional domain and one or more linker sequence linking the peptide and the functional domain.

Any functional domain suitable for an intended use may be fused to the peptides (and linkers) of the disclosure. In various non-limiting embodiments, the functional domain may comprise an immobilization domain, such as surface binding domain (solid binding domain) or a marker domain (for predictive, diagnostic, digital and prognostic purposes), a protein binding domain (for example, antibody binding), an optically adsorbing or color-changing or photoluminescent or fluorophore domain (e.g., GFP, BFP, YFP, CFP and their derivatives), a polymer or textile-specific binding domain, a second or further copy of a VOC binding peptide (identical or different), non-biological fluorophore domain (such as xanthene derivatives, e.g., fluorescein, rhodamine, Oregon green; pyrene derivatives, such as cascade blue, and oxazine derivatives such as Nile red, Nile blue, cresyl violet), or electro-magnetically active molecules, nanoparticles and quantum dots.

In some embodiments of the chimeric molecular construct, the one or more functional domains includes a surface (solid) binding domain. In some embodiments, the surface binding domain includes an amino acid sequence of at least 50%, 55%, 60%, 65%, 70%, 75%, 80%, 85%, 90%, 95%, or 100% identical to the amino acid sequence selected from the group consisting of SEQ ID NOS: 34-64 as shown in Table 1. In other embodiments, the surface binding domain includes an amino acid sequence at least 50%, 75%, or 100% identical to the amino acid sequence YSSY (SEQ ID NO:34). In some embodiments, present surface binding domain include one or more amino acid substitutions as compared to a reference or canonical sequence for the surface binding domain. In some embodiments, the one or more substitutions are conservative amino acid substitutions, while in other embodiments, all substitutions are conservative substitutions.

The surface binding domain can include any amino acid sequence of a peptide that can bind to any solid surface, Such binding peptides may, for example, be selected through directed evolution using, e.g., phage display peptide libraries. Table 1 provides a non-limiting list of surface binding domain sequences.

TABLE 1
Surface Binding Domains

| | | SEQ |
|---|---|---|
| | | ID |
| Name | Sequence | NO |
| GrBP5 | YSSY | 34 |
| Domain III | | |
| GrBP5 | IMVTESSDYSSY | 35 |
| GrBP5-M1 | IMVTESSDASSA | 36 |
| GrBP5-M2 | IMVTESSDWSSW | 37 |
| GrBP5-M3 | IMVTKSSRFSSF | 38 |
| GrBP5-M4 | TQSTKSSRYSSY | 39 |
| GrBP5-M5 | IMVTESSRYSSY | 40 |
| GrBP5-M6 | IMVTASSAYDDY | 41 |
| GrBP5-M7 | IMVTASSAYRDY | 42 |
| GrBP5-M8 | IMVTASSAYRRY | 43 |
| GrBP5-12 | IMVTASSDYSSY | 44 |
| HGrBPS | HIMVTESSDYSSY | 45 |
| WGrBPS | WIMVTESSDYSSY | 46 |
| VVGrBPS | VVIMVTESSDYSSY | 47 |
| SSGrBPS | SSIMVTESSDYSSY | 48 |
| GrBP5 | LIATESSDYSSY | 49 |
| hydrophobic | | |
| GrBP 5 | AQTTESSDYSSY | 50 |
| hydrophilic | | |
| GrBP 5 neutral | IMVTASSAYSSY | 51 |
| Bio-GrBP5 | (Biotin)-IMVTESSDYSSY | 52 |
| | ((Biotin)-N-terminal | |
| | α-amine) | |
| Rigid GrBP5 | IMVTEPPDYSSY | 53 |
| Cys-GrBP5 | CIMVTESSDYSSY | 54 |
| DOPA-GrBP5 | DOPA-IMVTESSDYSSY | 55 |
| | (DOPA-N-terminal a-amine) | |
| | (DOPA: dihydroxyphenylalanine) | |
| AminoF-GrBP5 | IMVTESSD(Nonnatural F)SSY | 56 |
| F-Phenyl- | IMVTESSD(F-Phenyl)SSY | 57 |
| GrBP5 | | |
| D-GrBP5 | IMVTESSDYSSY | 58 |

(all residues are D amino acids for D-GrBP5)

| | | |
|---|---|---|
| AntibodyBP- | EPIHRSTLTALLSSIMVTESSDYSSY | 60 |
| SS-GrBP5 | | |
| GrBP5 neutral | IMVTNSSNWSSW | 61 |
| WSSW | | |
| Seq 62 | IMVTESSDFSSF | 62 |
| Seq 63 | TQSTESSDYSSY | 63 |
| GRP5-M10 | IMVTDSSAYSSY | 64 |

Surface binding domains of the disclosure may bond to a surface by covalent or non-covalent bonding. In some embodiments, the non-covalent binding interaction is characterized as a hydrogen bond, an ionic bond, a van der Waals interaction, a hydrophobic interaction, a cation-pi interaction, a planar stacking interaction, or a metallic bond. In some embodiments, the surface binding domain bonds to a surface by non-covalent bonding, and in some embodiments, the surface binding domain bonds to the surface, at least in part, by a planar stacking interaction. As one example, aromatic residues such as tyrosine (Y) are known to strongly interact with graphitic surfaces through a coupling of π-electrons via planar stacking.

The one or more linker sequences in the chimeric molecular construct, when present, may be any amino acid linker as deemed appropriate for an intended use. In non-limiting embodiments, the linker may be a G-rich, an A-rich, or a GS-rich linker. In another embodiment, the linker is between 1-6 amino acids in length, or 1-5, 1-4, 1-3, 1-2, 2-6, 2-5, 2-4, 2-3, 3-6, 3-5, 3-4, 1, 2, 3, 4, 5, or 6 amino acids in length. In other embodiments, the linker may be G, GG, GGG GGGG, GGGGG, GGGGGG, GS(x) where x is 1-5; (G(x)S(y))z, where x, y and z are independently 1, 2, 3, 4, or 5; xP (1-6, or 3-6, or 4-6) where P is proline, or (EAAAK)n, where n is 1-5 (xP and (EAAAK)n represent exemplary rigid linkers); or xQ (3<x<6), where Q is glutamine.

In some embodiments, the chimeric molecular construct includes an amino acid sequence at least 50%, 55%, 60%, 65%, 70%, 75%, 80%, 85%, 90%, 95%, or 100% identical to the amino acid sequence of SEQ ID NOS: 11-32. As can be seen in Table 3, SEQ ID NO:11, SEQ ID NO:13, SEQ ID NO:15, SEQ ID NO: 17, SEQ ID NO:19, SEQ ID NO:21, SEQ ID NO: 23, SEQ ID NO:25, SEQ ID NO:27, SEQ ID NO:29, and SEQ ID NO:31 include a three amino acid linker “GGG” between the peptide and the surface binding domain. SEQ ID NO: 12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, and SEQ ID NO:32 include a linker labeled “X” that includes one of the linkers described above, or other amino acid sequence as deemed suitable for the intended use.

In some embodiments, the chimeric molecular construct includes one peptide, one functional domain and one linker sequence (1:1:1 constructs), with the linker sequence linking the peptide and the functional domain. In certain embodiments, each of the peptide, the functional domain and the linker in such embodiments are as described above, with the peptide an amino acid sequence at least 50%, 55%, 60%, 65%, 70%, 75%, 80%, 85%, 90%, 95%, or 100% identical to the amino acid sequence selected from SEQ ID NOS: 1-10 or SEQ ID NO: 33 and, in some embodiments, one or more conservative amino acid substitutions, or all conservative substitutions. In some embodiments of 1:1:1 constructs, the functional domains includes a surface binding domain, which in certain embodiments an amino acid sequence of at least 50%, 55%, 60%, 65%, 70%, 75%, 80%, 85%, 90%, 95%, or 100% identical to the amino acid sequence selected from the group consisting of SEQ ID NOS: 34-64, as well as any functional domain suitable for an intended use. Likewise, the linker includes one of the linkers described above, or otherwise suitable for the intended use.

In certain embodiments of the chimeric molecular construct, the functional domain includes one or more further copies of the peptides described above. As a non-limiting example of a functional domain being a surface binding domain, the surface binding domain is be fused to two or more peptides, each peptide independently connected to the surface binding domain either through a linker or, in the absence of a linker, directly. Once the surface binding domain is bound to a surface location, two or more VOC-binding peptides are immobilized at such location instead of one peptide, thus increasing the number and/or density of VOC binding sites on such surface.

As indicated above, in some embodiments of the chimeric molecular constructs described herein, linkers are absent. In such embodiments, the one or more peptides are fused directly to the functional domain.

In certain embodiments, the chimeric molecular constructs as provided above are described according to the formula X1-X2-X3, wherein X2 is an optional amino acid linker; and one of X1 and X3 is a functional domain, and the other is a peptide. In some embodiments, X1 is a peptide at least 50% identical to the amino acid sequence of SEQ ID NO:1 to SEQ ID NO: 10, or SEQ ID NO:33. In other embodiments, the peptide has an amino acid sequence at least 50% identical to SEQ ID NO:1, SEQ ID NO:3, SEQ ID NO:5, SEQ ID NO:6, SEQ ID NO: 8, SEQ ID NO:10, or SEQ ID NO:33. In other embodiments, the peptide includes an amino acid sequence at least 55%, 60%, 65%, 70%, 75%, 80%, 85%, 90%, 95%, or 100% identical to the amino acid sequence of SEQ ID NO:1 to SEQ ID NO:10, or SEQ ID NO:33, while in other embodiments, the peptide includes an amino acid sequence that is at least 55%, 60%, 65%, 70%, 75%, 80%, 85%, 90%, 95%, or 100% identical to the amino acid sequence of SEQ ID NO:1, SEQ ID NO:3, SEQ ID NO:5, SEQ ID NO:6, SEQ ID NO:8, SEQ ID NO: 10, or SEQ ID NO:33. X3 is a surface binding domain of an amino acid sequence of at least 50%, 55%, 60%, 65%, 70%, 75%, 80%, 85%, 90%, 95%, or 100% identical to the amino acid sequence selected from the group consisting of SEQ ID NOS: 34-64 as shown in Table 1. In other embodiments, X3 includes an amino acid sequence at least 50%, 75%, or 100% identical to the amino acid sequence YSSY (SEQ ID NO:34). Substitutions to a reference peptide sequence of X1 or X3 encompass those described elsewhere herein.

In some embodiments, the chimeric molecular construct of any aspect, embodiment or combination of embodiments of the disclosure is 50, 45, 40, 35, 30, 29, 28, 27, 26, 25, 24, 23, 22, 21, or 20 residues in length or less, or between 10-45, 10-40, 10-35, 10-29, 10-28, 10-27, 10-26, 10-25, 10-24, 10-23, 10-22, 10-21, or 10-20 amino acids in length.

In other embodiments, a chimeric molecular construct is provided including a first domain that binds to a VOC; and a second domain including two or more surface (solid) binding groups. In embodiments, the second domain is capable of non-covalent bonding to a solid, and in some embodiments, the second domain bonds to the surface, at least in part, by a planar stacking interaction. Amino acids that interact with a surface, contribute to surface bonding, or are held against the surface due to one or more adjacent amino acids interacting or contributing to surface bonding, are considered to be in “intimate contact” with the surface. A peptide, when bound to a surface, can have all amino acids in intimate contact with the surface. A peptide can also have a subset of amino acids in intimate contact with the surface, leaving the remaining amino acids to be tethered to the surface but not in intimate contact. In some embodiments, the chimeric molecular construct is capable of being brought into >50% intimate contact with the solid surface, and in some embodiments into >90% intimate contact with the solid surface.

In some embodiments of this aspect, as well as the other aspects of the disclosure including a peptide, a peptide includes one or more hydrophilic residues that causes some or all of the non-binding portions of the peptide to not be in substantial, or intimate, contact with the surface. In some embodiments, the peptide is a peptide construct that includes of a complex of multiple peptides, or a peptide complexed with one or more of: a metal atom or ion, a separate organic functional group or ion, a small molecule, a nanocrystal. In some embodiments of this aspect, as well as the other aspects of the disclosure, the peptide or chimeric molecular construct is complexed with one or more of: a metal atom or ion, a separate organic functional group or ion, a small molecule, and/or a nanocrystal.

In some embodiments, one or more chimeric molecular constructs is bound to a surface, and one or more additional surface binding peptides are bound to the same surface, wherein the additional surface binding peptides are not linked or fused to the chimeric molecular construct of the disclosure, and may be used, for example, to limit confounding signals or interactions on the surface, for example when used as a device active surface.

In some embodiments, the one or more additional surface binding peptides are shorter in length than the peptide or chimeric molecular construct, and, in some embodiments, include the same surface binding domain as the chimeric molecular construct. In some embodiments, the one or more additional solid-binding peptides includes the same linker sequence group as the chimeric molecular construct, and in some embodiments, the additional solid-binding peptides do not have a significant binding affinity to the target of the chimeric molecular construct. In certain embodiments, the one or more additional surface binding peptides have a higher hydrophobicity than the peptide, while in other embodiments, the one or more additional solid-binding peptides are more hydrophilic than the peptide. In embodiments, the solid-binding peptides act as inert, anti-fouling surface modification that prevent non-specific adsorption of interferant proteins, metabolites, and off-target species onto sensor surfaces while allowing specific detection of biomolecular targets.

### Compositions

In another aspect, a composition is provided including 1, 2, 3, 4, 5, 6, 7, 8, 9, 10, 11, 12 or more of the chimeric molecular constructs of the disclosure. In some embodiments, the chimeric molecular constructs include one or more peptides that binds to a volatile organic compound, fused to one or more functional domains and one or more linker sequence linking the peptide and the functional domain. In other embodiments, the one or more peptides of the chimeric molecular construct includes an amino acid sequence at least 50%, 55%, 60%, 65%, 70%, 75%, 80%, 85%, 90%, 95%, or 100% identical to the amino acid sequence selected from SEQ ID NOS: 1-10 or SEQ ID NO:33 fused to one or more functional domain and one or more linker sequence linking the peptide and the functional domain. In some embodiments the one or more functional domains includes a surface binding domain. In some embodiments, the surface binding domain includes an amino acid sequence of at least 50%, 55%, 60%, 65%, 70%, 75%, 80%, 85%, 90%, 95%, or 100% identical to the amino acid sequence selected from the group consisting of SEQ ID NOS: 34-64 as shown in Table 1. In other embodiments, the surface binding domain includes an amino acid sequence at least 50%, 75%, or 100% identical to the amino acid sequence YSSY (SEQ ID NO:34). In some embodiments, the one or more linkers includes a G-rich, an A-rich, or a GS-rich linker between 1-6 amino acids in length, or 1-5, 1-4, 1-3, 1-2, 2-6, 2-5, 2-4, 2-3, 3-6, 3-5, 3-4, 1, 2, 3, 4, 5, or 6 amino acids in length; G, GG, GGG GGGG, GGGGG, GGGGGG; GS(x) where x is 1-5; (G(x)S(y))z, where x, y and z are independently 1, 2, 3, 4, or 5; xP (1-6, or 3-6, or 4-6) where P is proline, or (EAAAK)n, where n is 1-5; or xQ (3<x<6), where Q is glutamine.

In some embodiments, the composition includes 1, 2, 3, 4, 5, or all 6 of the following: (a) at least one amino acid sequence at least 50%, 55%, 60%, 65%, 70%, 75%, 80%, 85%, 90%, 95%, or 100% identical to the amino acid sequence of SEQ ID NOS: 11-12; (b) at least one amino acid sequence at least 50%, 55%, 60%, 65%, 70%, 75%, 80%, 85%, 90%, 95%, or 100% identical to the amino acid sequence of SEQ ID NOS: 13-14; (c) at least one amino acid sequence at least 50%, 55%, 60%, 65%, 70%, 75%, 80%, 85%, 90%, 95%, or 100% identical to the amino acid sequence of SEQ ID NOS: 15-16; (d) at least one amino acid sequence at least 50%, 55%, 60%, 65%, 70%, 75%, 80%, 85%, 90%, 95%, or 100% identical to the amino acid sequence of SEQ ID NOS: 17-18; (e) at least one amino acid sequence at least 50%, 55%, 60%, 65%, 70%, 75%, 80%, 85%, 90%, 95%, or 100% identical to the amino acid sequence of SEQ ID NOS: 19-20; and (f) at least one amino acid sequence at least 50%, 55%, 60%, 65%, 70%, 75%, 80%, 85%, 90%, 95%, or 100% identical to the amino acid sequence of SEQ ID NOS: 21-22. Such embodiments can be capable of detecting one or more VOCs common to COVID-19 infection as described elsewhere herein.

In some embodiments, the composition includes the 1, 2, 3, 4, 5, or all 6 sequences described directly above, further including 1, 2, 3, 4, or all 5 of the following: (a) at least one amino acid sequence at least 50%, 55%, 60%, 65%, 70%, 75%, 80%, 85%, 90%, 95%, or 100% identical to the amino acid sequence of SEQ ID NOS: 23-24; (b) at least one amino acid sequence at least 50%, 55%, 60%, 65%, 70%, 75%, 80%, 85%, 90%, 95%, or 100% identical to the amino acid sequence of SEQ ID NOS: 25-26; (c) at least one amino acid sequence at least 50%, 55%, 60%, 65%, 70%, 75%, 80%, 85%, 90%, 95%, or 100% identical to the amino acid sequence of SEQ ID NOS: 27-28; (d) at least one amino acid sequence at least 50%, 55%, 60%, 65%, 70%, 75%, 80%, 85%, 90%, 95%, or 100% identical to the amino acid sequence of SEQ ID NOS: 29-30; and (e) at least one amino acid sequence at least 50%, 55%, 60%, 65%, 70%, 75%, 80%, 85%, 90%, 95%, or 100% identical to the amino acid sequence of SEQ ID NOS: 31-32. Such embodiments can be capable of detecting one or more VOCs common to diseases or disorders confounding to detection of COVID-19 infection as described elsewhere herein.

Compositions as described above and herein can bind one or more VOCs and may be capable of being bound to a surface. Compositions with two or more chimeric molecular constructs may be, e.g., mixed together and bound to a surface, bound separately in discrete regions of a surface, or separately bound to different surfaces. In some embodiments including two or more chimeric molecular constructs, the constructs are separately bound to discrete surface regions on a single device (e.g., a transistor such as a field-effect transistor), enabling concurrent probing of a sample for separate VOCs.

In some embodiments, the 1, 2, 3, 4, 5, 6, 7, 8, 9, 10, 11, 12, or more of the chimeric molecular constructs in a composition are bound to a surface, which bonding, in embodiments, is non-covalent. In some embodiments, the surface includes a carbonaceous surface, and the chimeric molecular constructs are non-covalently bound to the carbonaceous surface. In some embodiments, the carbonaceous surface includes a carbon nanotube, including but not limited to a single wall carbon nanotube or multiwall carbon nanotube. In other embodiments, the carbonaceous surface includes a single or multilayer graphene or graphitic carbon surface.

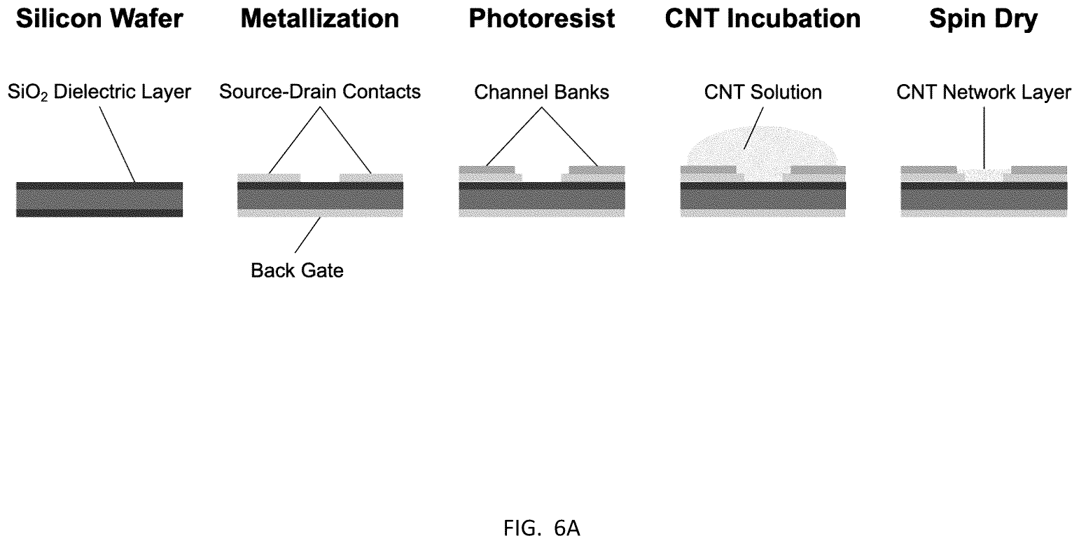

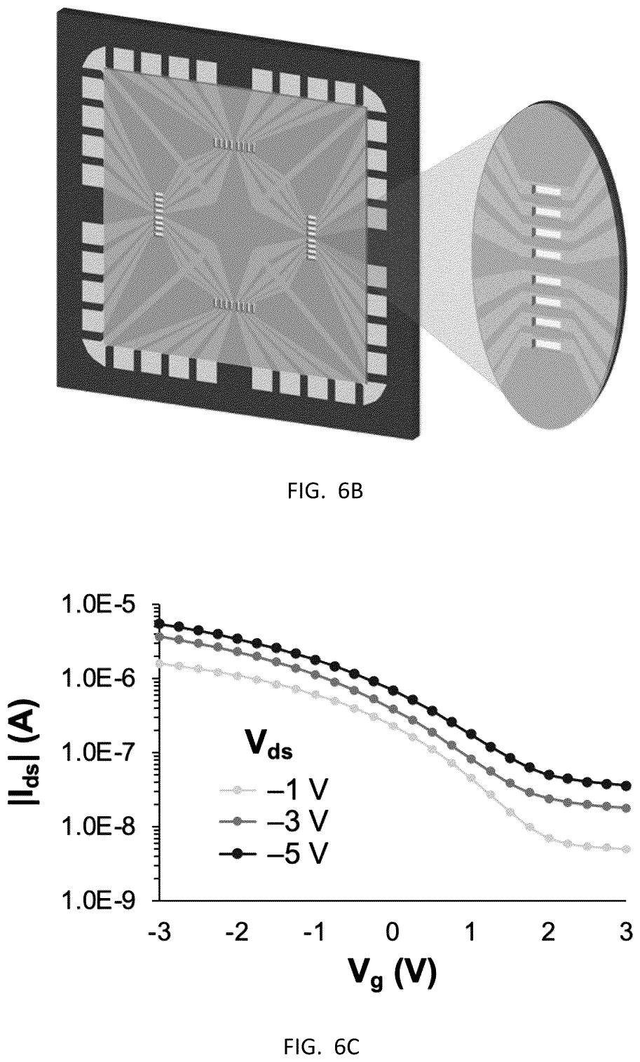

Multiple molecular targets can be detected in a sample using these compositions of peptide or chimeric molecular constructs, immobilized onto a surface of a diagnostic device (e.g., in an array; see FIG. 6B). Such embodiments can be referred to as multiplexed sensors or devices. One embodiment of a multiplexed device is called COVIDalyzer™, in which case the target molecules are volatile organic compounds that are exclusively generated by COVID-19 patients. In embodiments of the COVIDalyzer, one or more peptides of Table 2, or chimeric molecular constructs according to SEQ ID NOS: 11, 13, 15, 17, 19, 21, 23, 25, 27, 29, or 31 are bound to a device active surface of the COVIDalyzer.

Surfaces, as described herein, generally refer to functional surfaces, or surfaces that can enable detection of, e.g., a binding event. A binding event can include a peptide, chimeric molecular construct or composition including a chimeric molecular construct, immobilized on the surface, binding to its molecular target(s), e.g., a VOC. In some embodiments, the surface is included as part of a sensor and/or diagnostic device and referred to as a device active surface or sensing surface.

The surface may be any surface as deemed appropriate for an intended use. In various non-limiting embodiments, the surface may comprise a solid surface in a variety of geometries such as bulk, nanoparticles, nanorods, nanowires, nanotubes, single atomic layer solids, such as MoS₂, WSe₂, graphene, glass (glass bead, micro and nanofluidic channels), ceramics (functional ceramics such as conducting and magnetic), polymers (thermoset or thermoplastic, such as silicone, nylon, bakelite), biologicals (lipid membranes, polysaccharides, viral and cell surface), carbonaceous surfaces (e.g., graphite, graphene, diamond, carbon nanotubes, carbon black and as otherwise defined herein), and functionalized derivatives thereof.

In some embodiments, the surface is a one dimensional semiconducting element, such as a ZnO, ZnO derivatives, or Si nanowire, where the one-dimensional semiconducting element has an aspect ratio >1 in cross section. In some embodiments, the surface includes of single-layer or multilayer transition metal dichalcogenides MX₂ (e.g., M=Mo, W and X=S, Se), and in some embodiments, the surface comprises a two-dimensional semiconductor, including but not limited to MoS₂, MoSe₂, MoTe₂, WS₂ and WSe₂, chalcogenide or III-V semiconductor. In yet other embodiments, the surface includes an oxide, II-VI, III-V or group IV bulk or thin film semiconductor, including but not limited to ZnO and its derivatives, Zn·S, ZnSe, ZnTe, CdS, CdSe, CdTe, GaAs, Si, and Ge. In other embodiments, the surface includes a semiconductor or semimetal including but not limited to a polymer semiconductor, a soluble small molecule semiconductor, a conducting polymer, a perovskite semiconductor. In yet other embodiments, the surface includes a dielectric or protective layer and target-binding peptides are bound to this dielectric or protective layer.

In some embodiments, the surface includes a two dimensional semiconducting element, an oxide, II-VI, III-V or group IV bulk or thin film semiconductor, a semiconductor or semimetal, or a dielectric or protective layer.

In some embodiments, the compositions described above and herein include a plurality of transistors, in which a single peptide or chimeric molecular construct is immobilized on a surface of each transistor in the plurality of transistors, and, in some embodiments, each transistor includes a different peptide or chimeric molecular construct.

In embodiments, the transistor includes a carbon nanotube field-effect transistors (CNT-FET), which uses carbon nanotubes as the active surface, or a graphene field effect transistor device (gFET), which uses graphene as the active surface.

In some embodiments, the compositions described herein comprise a CNT-FET or a gFET and, in some embodiments both CNT-FET and gFET. Such CNT-FETs and gFETs include one or more copies of a peptide or chimeric molecular construct of a single sequence (uniplex/monoplex), or one or more copies of peptides or chimeric molecular constructs of two or more different sequences as described herein (a multiplex arrangement). In some embodiments, multiplexed devices may also include single atomic layer solids, where the active device material can be e.g., MoS2, MoSe2, and the like.

When using single atomic layer solids as an active device surface, the binding of the surface binding (or X3) domain can be through a non-covalent, weak molecular interaction, ensuring the formation of coherent binding of the peptide onto the solid surface to avoid binding sites acting as defects, causing detrimental effects on device performance, such as mobility of electrons in the device. Exemplary validation of the formation of coherent interface formation between a chimeric molecular construct and the solid surface has been performed using HOPG (highly oriented pyrolytic graphite) and atomic force microscopy (AFM).

Immobilization and interrogation of peptides and chimeric molecular constructs on several surfaces is demonstrated in the examples below. For instance, carbon nanotubes, highly oriented pyrolytic graphite, bare gold, carbon coated gold, and polymer coated gold surfaces are used to detect binding of target molecules by the immobilized chimeric molecular constructs and to verify binding of the chimeric molecular constructs to the surface utilizing, e.g., CNT-FET sensor measurement, surface plasmon resonance, quartz crystal microbalance, and atomic force microscopy.

### Field Effect Transistors

In certain embodiments, the surface to which peptides and chimeric molecular constructs bind can be considered a sensing surface and can be included as part of a sensor or diagnostic device. In some embodiments, the sensor utilizes one or more conductance sensors on a substrate platform, wherein the sensor can be functionalized (bound) with peptides, chimeric molecular constructs or compositions thereof for binding the target analytes, e.g., VOCs. In certain embodiments, the peptides and chimeric molecular constructs or compositions thereof comprise natural polymers, synthetic polymers, antibodies, or small molecules.

In certain embodiments, the sensor can be made comprising carbon nanotubes or graphene. Graphene is a flat monolayer of carbon atoms tightly packed into a two-dimensional honeycomb lattice, while carbon nanotubes are single wall (one carbon layer) or multi-wall tubes made from carbon. In certain embodiments, the FET sensing element with CNTs or graphene as the conducting channel has an electric resistance of about 0.1 kΩ to about 30 kΩ. In certain embodiments, the FET sensing element with CNTs or graphene as the conducting channel has an electric resistance of about 0.1 kΩ to about 15 kΩ. In certain embodiments, the FET sensing element with CNTs or graphene as the conducting channel has an electric resistance of about 0.1 kΩ to about 10 kΩ. In certain embodiments, the FET sensing element with CNTs or graphene as the conducting channel has an electric resistance of about 0.1 kΩ to about 5 kΩ, and in certain embodiments, the FET sensing element with CNTs or graphene as the conducting channel has an electric resistance of about 0.1 kΩ to about 3 kΩ. In certain embodiments, CNT or graphene as the conductance channel has an electric resistance of about 0.1 kΩ to about 3 kΩ, about 0.25 kΩ to about 2.75 kΩ, about 0.5 kΩ to about 2.5 kΩ, about 0.75 kΩ to about 2.25 kΩ, about 1 kΩ to about 2 kΩ, about 1.25 kΩ to about 1.75 kΩ, about 1.5 kΩ to about 2 or about 2 kΩ to about 3 kΩ. In certain embodiments, CNT or graphene as the conductance channel has an electric resistance of at least about 0.1 kΩ, at least about 0.2 kΩ, at least about 0.3 kΩ, at least about 0.4 kΩ, at least about 0.5 kΩ, at least about 0.6 kΩ, at least about 0.7 kΩ, at least about 0.8 kΩ, at least about 0.9 kΩ, at least about 1 kΩ, at least about 1.2 kΩ, at least about 1.4 kΩ, at least about 1.6 kΩ, at least about 1.8 kΩ, at least about 2 kΩ, at least about 2.2 kΩ, at least about 2.4 kΩ, at least about 2.6 kΩ, at least about 2.8 kΩ, at least about 3 kΩ, at least about 4 kΩ, at least about 5 kΩ, at least about 6 kΩ, at least about 7 kΩ, at least about 8 kΩ, at least about 9 kΩ, at least about 10 kΩ, at least about 15 kΩ, at least about 20 kΩ, at least about 25 kΩ, or about 30 kΩ. In certain embodiments, CNT or graphene as the conductance channel has an electric resistance of no more than about 0.1 kΩ, no more than about 0.2 kΩ, no more than about 0.3 kΩ, no more than about 0.4 kΩ, no more than about 0.5 kΩ, no more than about 0.6 kΩ, no more than about 0.7 kΩ, no more than about 0.8 kΩ, no more than about 0.9 kΩ, no more than about 1 kΩ, no more than about 1.2 kΩ, no more than about 1.4 kΩ, no more than about 1.6 kΩ, no more than about 1.8 kΩ, no more than about 2 kΩ, no more than about 2.2 kΩ, no more than about 2.4 kΩ, no more than about 2.6 kΩ, no more than about 2.8 kΩ, no more than about 3 kΩ, no more than about 4 kΩ, no more than about 5 kΩ, no more than about 6 kΩ, no more than about 7 kΩ, no more than about 8 kΩ, no more than about 9, no more than about 10 kΩ, no more than about 15 kΩ, no more than about 20 kΩ, no more than about 25 kΩ, or no more than about 30 kΩ.

In certain embodiments, the graphene sensor includes a single layer sheet. In certain embodiments, the graphene sensor includes a multilayered sheet. In certain embodiments, the graphene sensor includes at least one layer of graphene. In certain embodiments, the graphene sensor includes at least two layers of graphene, at least three layers of graphene, or at least four layers of graphene. In certain embodiments, the graphene sheet can be formed by mechanical exfoliation, chemical exfoliation, chemical vapor deposition, or silicon carbide.

In certain embodiments, the CNT sensor includes a disordered or random array of CNTs. In certain embodiments, the CNT sensor includes an ordered array of CNTs. In certain embodiments, the CNTs are dissolved in a solvent and drop deposited. In some embodiments, the CNTs are printed in place with CNT-based ink.

The sensors as disclosed herein can be highly sensitive, as changes in surface charge due to the presence of the target analyte near the sensor or the target analyte binding leads to a detectable signal even at low target analyte concentrations.

In certain embodiments, the sensor can be constructed on a substrate platform. In certain embodiments, the substrate can be fabricated on the final substrate. In certain embodiments, the substrate can be fabricated on a thin flexible film and the sensor device (including the thin flexible film) and attached to the final substrate platform. In certain embodiments, the substrate platform and the thin layer film can be made from the same material. In certain embodiments, the substrate platform and thin later film can be made from different material. In certain embodiments, the sensor can be formed on a substrate platform that is then formed/molded (e.g., by heating) into a desired shape.

A set of peptides or chimeric molecular constructs immobilized on the conductance sensor can bind to the target analyte. In certain embodiments, the peptides or chimeric molecular constructs can bind reversibly or irreversibly to the target analyte. In certain embodiments, the peptides or chimeric molecular constructs can bind reversibly to the target analyte. In certain embodiments, the peptides or chimeric molecular constructs can bind to more than one target analyte.

In certain embodiments, target analyte binding of the peptides or chimeric molecular constructs can change the charge density on the sensor surface, inducing changes in the carrier concentration of the sensor.

The substrate or platform surface can be one or multi-layered. For example, the substrate or platform surface can include two layers (see FIG. 6A) such as, but not limited to a silicon wafer based device. An example of a silicon wafer based device, the lower layer can be silicon as the substrate while the upper later is silicone oxide which can serve as an insulating layer. In certain embodiments, the substrate platform can be a single layer. In certain embodiments the single later substrate platform can be s a polymer substrate.

In certain embodiments, the electrode wire can be, for example, but not limited to Ag/AgCl, Ag, Pt, or combinations thereof.

In certain embodiments, the dielectric layer can be made from material such as, but not limited to SiO2, hexagonal boron nitride (h-BN), HfO2, parylene, Si3N4, or combinations thereof.

In certain embodiments, the gate electrode can be made from material such as, but not limited to ITO, Ti/Pd/Pt, gold, copper, chromium, or mixtures thereof.

In certain embodiments, the source and drain electrodes, can be separately made from material such as, but not limited to ITO, Ti/Pd/Pt, chromium, gold, chromium, or combinations thereof.

In certain embodiments, the polymer substrate and or thin layer film can be made from material such as, but not limited to polyethylene terephthalate (PET), polycarbonate polystyrene, polymethyl methacrylate (PMMA), polymacon, silicones, fluoropolymers, silicone acrylate, fluoro-silicone/acrylate, poly hydroxyethyl methacrylate, or combinations thereof.

In certain embodiments, the dimensions of the CNT or graphene conducting channel can be a length of about 150 μm to about 250 μm by a width of about 10 μm to about 100 μm by a thickness of about 1 μm to about 10 nm. In certain embodiments, the dimensions of the CNT or graphene conducting channel can be a length of about 1 μm to about 50 μm by a width of about 50 μm to about 150 μm by a thickness of about 1 μm to about 10 nm. In certain embodiments, the CNT or graphene conducting channel can be functionalized with a peptide, chimeric molecular construct or compositions thereof.

In certain embodiments, the length of a CNT or graphene channel in the sensor can be about 150 μm to about 250 μm, about 160 μm to about 240 μm, about 170 μm to about 230 μm, about 180 μm to about 220 μm, about 190 μm to about 210 μm, about 150 μm to about 240 μm, about 150 μm to about 230 μm, about 150 μm to about 220 μm, about 150 μm to about 210 μm, about 150 μm to about 200 μm, about 150 μm to about 190 μm, about 150 μm to about 180 μm, about 150 μm to about 170 μm, about 160 μm to about 250 μm, about 170 μm to about 250 μm, about 180 μm to about 250 μm, about 190 μm to about 250 μm, about 200 μm to about 250 μm, about 210 μm to about 250 μm, about 220 μm to about 250 μm, about 230 μm to about 250 μm, or about 240 μm to about 250 μm. In certain embodiments, the width of a CNT or graphene channel in a sensor can be about 10 μm to about 100 μm, about 20 μm to about 90 μm, about 30 μm to about 80 μm, about 40 μm to about 70 μm, about 50 μm to about 60 μm, about 10 μm to about 100 μm, about 10 μm to about 90 μm, about 10 μm to about 80 μm, about 10 μm to about 70 μm, about 10 μm to about 60 μm, about 10 μm to about 50 μm, about 10 μm to about 40 μm, about 10 μm to about 30 μm, about 10 μm to about 20 μm, about 10 μm to about 100 μm, about 30 μm to about 100 μm, about 40 μm to about 100 μm, about 50 μm to about 100 μm, about 60 μm to about 100 μm, about 70 μm to about 100 μm, about 80 μm to about 100 μm, or about 90 μm to about 100 μm.

In certain embodiments, the length of a CNT or graphene channel in the sensor can be about 1 μm to about 50 μm, about 5 μm to about 40 μm, about 10 μm to about 35 μm, about 15 μm to about 30 μm, about 20 μm to about 30 μm, about 25 μm, about 1 μm to about 50 μm, about 1 μm to about 40 μm, about 1 μm to about 35 μm, about 1 μm to about 30 μm, about 1 μm to about 25 μm, about 1 μm to about 20 μm, about 1 μm to about 15 μm, about 1 μm to about 10 μm, about 1 μm to about 5 μm, about 5 μm to about 50 μm, about 10 μm to about 50 μm, about 15 μm to about 50 μm, about 20 μm to about 50 μm, about 25 μm to about 50 μm, about 30 μm to about 50 μm, about 35 μm to about 50 μm, about 40 μm to about 50 μm, or about 45 μm to about 50 μm. In certain embodiments, the width of a CNT or graphene channel in a sensor can be about 50 μm to about 150 μm, about 60 μm to about 140 μm, about 70 μm to about 130 μm, about 80 μm to about 120 μm, about 90 μm to about 110 μm, about 100 μm, about 50 μm to about 140 μm, about 50 μm to about 130 μm, about 50 μm to about 120 μm, about 50 μm to about 110 μm, about 50 μm to about 100 μm, about 50 μm to about 90 μm, about 50 μm to about 80 μm, about 50 μm to about 70 μm, about 50 μm to about 60 μm, about 60 μm to about 150 μm, about 70 μm to about 150 μm, about 80 μm to about 150 μm, about 90 μm to about 150 μm, about 100 μm to about 150 μm, about 110 μm to about 150 μm, about 120 μm to about 150 μm, about 130 μm to about 150 μm, or about 140 μm to about 150 μm.

### Methods

The multiplexed detection methods of the disclosure generally involve assessing the molecular signature of multiple molecular targets (e.g., VOC) in a mixture that defines the presence or progression of a disease or disorder, where the molecular targets in a mixture can be detected using a set of molecular probes designed for this purpose, each of which having an affinity to a particular target potentially present in the mixture, all immobilized on the same or different sensor units, resulting in detection of molecular targets in the mixture that define a specific molecular signature, created using machine learning algorithm developed for this purpose (as described in the examples).

In another aspect, the disclosure provides a method for detecting, prognosing, or monitoring treatment for COVID-19 infection, the method including contacting with a biological sample a composition of the disclosure as described above and herein; detecting volatile organic compounds in the biological sample by their binding to the peptides or chimeric molecular constructs present in the composition; and comparing a signature of VOCs present in the biological sample with a VOC signature characteristic of COVID-19; wherein a VOC signature present in the sample that matches a VOC signature characteristic of COVID-19 serves to detect COVID-19 infection, prognosis, or response to treatment in the biological sample.

The disclosure also provides a method for detecting, prognosing, or monitoring treatment for COVID-19 infection in the presence of one or more non-COVID-19 respiratory infection (such as variants of common cold, influenza, and Streptococcus), cancer of the bowel, airways, or lungs, asthma, chronic obstructive pulmonary disease (COPD), epileptic seizure, or any human or animal disease presenting a detectable VOC signature, including contacting with a biological sample a composition of the disclosure as described above and herein; detecting volatile organic compounds (VOCs) in the biological sample by their binding to the peptides or chimeric molecular constructs present in the composition; and comparing a signature of VOCs present in the biological sample with a VOC signature characteristic of COVID-19; wherein a VOC signature present in the sample that matches a VOC signature characteristic of COVID-19 serves to detect COVID-19 infection, prognosis, or response to treatment in the biological sample.

In some embodiments of these methods, the composition includes 1, 2, 3, 4, 5, 6, 7, 8, 9, 10, 11, 12, or more of the chimeric molecular constructs of the disclosure, or channels. In other embodiments, the composition includes 1, 2, 3, 4, 5, 6, 7, 8, 9, 10, 11, 12, or more chimeric molecular constructs that include one or more peptides that binds to a volatile organic compound, fused to one or more functional domains and one or more linker sequence linking the peptide and the functional domain.

In other embodiments of these methods, the composition includes 1, 2, 3, 4, 5, or all 6 of the following: (a) at least one amino acid sequence at least 50%, 55%, 60%, 65%, 70%, 75%, 80%, 85%, 90%, 95%, or 100% identical to the amino acid sequence of SEQ ID NOS: 11-12; (b) at least one amino acid sequence at least 50%, 55%, 60%, 65%, 70%, 75%, 80%, 85%, 90%, 95%, or 100% identical to the amino acid sequence of SEQ ID NOS: 13-14; (c) at least one amino acid sequence at least 50%, 55%, 60%, 65%, 70%, 75%, 80%, 85%, 90%, 95%, or 100% identical to the amino acid sequence of SEQ ID NOS: 15-16; (d) at least one amino acid sequence at least 50%, 55%, 60%, 65%, 70%, 75%, 80%, 85%, 90%, 95%, or 100% identical to the amino acid sequence of SEQ ID NOS: 17-18; (e) at least one amino acid sequence at least 50%, 55%, 60%, 65%, 70%, 75%, 80%, 85%, 90%, 95%, or 100% identical to the amino acid sequence of SEQ ID NOS: 19-20; and (f) at least one amino acid sequence at least 50%, 55%, 60%, 65%, 70%, 75%, 80%, 85%, 90%, 95%, or 100% identical to the amino acid sequence of SEQ ID NOS: 21-22.

In some embodiments of these methods, the composition further includes 1, 2, 3, 4, or all 5 of the following: (a) at least one amino acid sequence at least 50%, 55%, 60%, 65%, 70%, 75%, 80%, 85%, 90%, 95%, or 100% identical to the amino acid sequence of SEQ ID NOS: 23-24; (b) at least one amino acid sequence at least 50%, 55%, 60%, 65%, 70%, 75%, 80%, 85%, 90%, 95%, or 100% identical to the amino acid sequence of SEQ ID NOS: 25-26; (c) at least one amino acid sequence at least 50%, 55%, 60%, 65%, 70%, 75%, 80%, 85%, 90%, 95%, or 100% identical to the amino acid sequence of SEQ ID NOS: 27-28; (d) at least one amino acid sequence at least 50%, 55%, 60%, 65%, 70%, 75%, 80%, 85%, 90%, 95%, or 100% identical to the amino acid sequence of SEQ ID NOS: 29-30; and (e) at least one amino acid sequence at least 50%, 55%, 60%, 65%, 70%, 75%, 80%, 85%, 90%, 95%, or 100% identical to the amino acid sequence of SEQ ID NOS: 31-32.

In some embodiments of these methods, the composition includes (a) at least one amino acid sequence of SEQ ID NOS: 11-12; (b) at least one amino acid sequence of SEQ ID NOS: 13-14; (c) at least one amino acid sequence of SEQ ID NOS: 15-16; (d) at least one amino acid sequence of SEQ ID NOS: 17-18; (e) at least one amino acid sequence of SEQ ID NOS: 19-20; and (f) at least one amino acid sequence of SEQ ID NOS: 21-22. In other embodiments, the composition further includes (a) at least one amino acid sequence of SEQ ID NOS: 23-24; (b) at least one amino acid sequence of SEQ ID NOS: 25-26; (c) at least one amino acid sequence of SEQ ID NOS: 27-28; (d) at least one amino acid sequence of SEQ ID NOS: 29-30; and (e) at least one amino acid sequence of SEQ ID NOS: 31-32.

In another embodiment of these methods, the composition includes (a) at least one amino acid sequence of SEQ ID NO:11; (b) at least one amino acid sequence of SEQ ID NO: 13; (c) at least one amino acid sequence of SEQ ID NO:15; (d) at least one amino acid sequence of SEQ ID NO:17; (e) at least one amino acid sequence of SEQ ID NO:19; and (f) at least one amino acid sequence of SEQ ID NO:21. In other embodiments, the composition further includes (a) at least one amino acid sequence of SEQ ID NO:23; (b) at least one amino acid sequence of SEQ ID NO:25; (c) at least one amino acid sequence of SEQ ID NO:27; (d) at least one amino acid sequence of SEQ ID NO:29; and (e) at least one amino acid sequence of SEQ ID NO:31.

In some embodiments of these methods, the comparing step includes use of a computational model trained to differentiate biological samples with and without the VOC signature characteristic of COVID-19.

In embodiments of these methods, the chimeric molecular constructs are bound to a carbonaceous surface, which, in some embodiments, includes a carbon nanotube, including but not limited to a single wall carbon nanotube or multiwall carbon nanotube. And in some embodiments, the surface comprises a carbon nanotube field-effect transistors (CNT-FET), or a graphene field effect transistor (gFET).

For the methods described herein, any suitable biological sample from a subject to be tested may be used, including but not limited to exhaled breath sample, saliva sample, nasal or other mucus membrane swab, sweat or skin-contact sample, stool sample, urine sample, worn clothing sample, or other individual bodily fluid or tissue sample, as well as ambient air or other sample gathered from common areas, or any environment where VOCs of human or animal biological origin are present. In some embodiments, the biological sample is a breath sample. In embodiments, the biological sample is obtained from a subject at risk of COVID-19, including but not limited to a subject presenting acute respiratory symptoms, fever, or other symptoms (diarrhea, anosmia) indicative of COVID-19 infection, or who may have had exposure to or contact with confirmed or probable COVID-19 patients, or visited areas where transmission of SARS-COV-2 virus has been confirmed or suspected; or a subject that is being treated for COVID-19.

In some method embodiments, detecting non-VOC molecules is provided. Non-VOC molecules can include one or more proteins, protein fragments, peptides, or nucleic acids.

A variety of sensing modalities can be used for the detection of interaction between the target molecules and the peptides or chimeric molecular constructs of the disclosure, including electronic, magnetic, photonic, acoustic, colorimetric, optical, or other metric signal depending on the solid-state materials used in the active device with a specific device construction including the multiplexed architectures. As such, for some embodiments of the various methods described herein, the VOC signature present in the sample is converted to an electrical, photonic, magnetic, acoustic, colorimetric, optical, or other metric signal for the comparing step.

In another aspect, the disclosure provides a method for designing a peptide multiplex probe for detection of a volatile organic compound (VOC) signature in a biological or simulated biological sample (collectively referred to as “biological sample”), including providing a biological sample VOC signature to be detected and identifying a plurality of odorant-binding proteins (OBPs) or olfactory receptors (ORs) capable of detecting VOCs in the biological sample VOC signature; and training a computational model, using the VOC signature to be detected and the plurality of OBPs or ORs capable of detecting VOCs in the biological sample VOC signature, to perform functions including: (i) via an OBP or OR database, identifying a most closely-matching OBP or OR structure and identifying ligand-binding residues in each OBP or OR in the plurality of OBPs or ORs; (ii) extracting the ligand-binding amino acid residues in each OBP or OR and rearranging the order of extracted amino acid residues to mimic their arrangement in space in the OBP or OR, generating candidate peptide multiplex probes; (iii) testing the candidate peptide multiplex probes against the biological sample VOC signature to be detected, via a machine learning model trained to differentiate biological samples with and without the VOC signature to be detected; and (iv) selecting those candidate peptide multiplex probes that best differentiate biological samples with and without the VOC signature to be detected. In some embodiments, steps (b) (ii) and (b) (iii) are carried out a plurality of times, and, in some embodiments, molecular dynamics simulation techniques are used to differentiate biological samples with and without the VOC signature to be detected.

In some embodiments, a traveling salesmen algorithm can be used to rearrange the order of extracted amino acid residues to mimic their arrangement in space in the OBP or OR, and in some embodiments, molecular dynamics simulation techniques are used to rearrange the order of extracted amino acid residues.

In some embodiments, the computational model can include a model or an ensemble of multiple models working in parallel, and may employ one or more of the methods or architectures including, but no limited to: a neural network, a linear or kernel-modified linear regression model, a k-nearest-neighbors model, a decision tree or random forest of decision trees, a support vector classifier, a Bayesian inference model, a Markov decision process or other graphical decision model, an expert-designed heuristic model, and any of the proceeding models with parameters selected by a combination of gradient back-propagation, gradient descent, hill-climbing, random search, Bayesian search, and the like. As described in the examples and methods sections below, the methods can utilize neural networks for molecular machine learning tasks, or pretrained networks and models trained on a wide range of peptide-binding datasets to transfer, learn and create highly selective multiplexed sensors with reliable false positive rejection allowing for rapid fine-tuning of probe designs with a relatively small number of datapoints from the experimental data, while simultaneously growing a library of datasets and trained models.

As described in the examples and methods sections, the methods permit design of peptide multiplex probes that detect VOC-binding signatures for maximal differentiation between different VOC signatures (or “profiles”), where signatures may be, for example, profiles arising from multiple human diseases, simulated signatures, or randomly or otherwise generated VOC signatures representing unknown or undiscovered confounding VOC signals.

Sensitive and selective target-binding peptide molecular sequences are derived and refined through, e.g., data mining of binding affinity data from VOC odorant binding protein (OBPs) literature or de novo research, and using multiple-homology protein structure mapping. Though data pertaining to insect OBPs served as the primary source information for generation of VOC-binding peptides, other olfactory proteins are suitable for the methods described herein, including mammalian OBPs, mammalian olfactory receptors (ORs), other general VOC-binders, and the like. In some embodiments of the method for designing a peptide multiplex probe, the VOC binding moieties comprise one or more of insect OBPs, mammalian OBPs, olfactory receptors, originate is from a source other than OBPs, including, for example, mammalian olfactory receptors, or other peptide that can bind a VOC of interest.

### Electronics

In another aspect, the disclosure provides a non-transitory computer readable medium storing instructions that, when executed by a computing device, cause the computing device to perform any of the methods described herein. A computing device is also provided including one or more processors; and a computer readable medium storing instructions that, when executed by the one or more processors, cause the computing device to perform a method described herein. The computing device can be comprised by a diagnostic device, e.g., a hand-held VOC analyzer (e.g., COVIDalyzer) or benchtop or free standing POC VOC analyzer.

Quantitative analysis of a monoplex or multiplexed device may, for example, be based on a machine learning (ML)/artificial intelligence (AI) based algorithms on, e.g., point of care (POC) diagnostic devices used in home-based settings, field hospitals and clinics, permanent hospitals and clinics, and the like, each potentially having appropriate levels of sophistication of construction and analytical detection sensitivity, specificity and precision, as required. In some embodiments, the compositions may include devices that are electronically connected to relevant health monitoring centers to enable telemedicine for rapid diagnostics, prognostics, and treatment.

### Nucleic Acids, Expression Vectors and Host Cells

In another aspect the disclosure provides nucleic acids encoding the peptide, chimeric molecular construct or chimeric molecular construct of any aspect, embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded peptide or chimeric molecular construct, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the peptide or chimeric molecular construct of the disclosure.

In a further aspect, the disclosure provides expression vectors comprising the nucleic acid of any aspect or embodiment of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids or expression vectors (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

For disease or disorder diagnosis, a differentiating feature enabled by the technology of the disclosure is the detection of VOCs and, optionally, non-VOC molecules (e.g., cytokines, cell-free DNA), which distinguishes the embodiments disclosed herein from existing qPCR methods that rely solely on extracted RNA, from immunoassays that rely on antibodies produced by the immune response, and from mass spectroscopy-based or gas chromatography-based approaches that are typically used with volatile organics. By targeting various types of biomarkers, sensors of the disclosure can capture the entire spectrum of molecules exhaled by a human, non-human mammal or non-mammalian animal patient, thereby allowing for a complete “breathomics” approach to COVID-19 diagnosis. Selectivity for biomarker targets is enhanced using bioinformatics-driven rational design with respect to, e.g., VOC detection using peptides derived from olfactory and gustatory receptors, as well as metabolic enzymes (e.g., alcohol dehydrogenase), that are specific to VOC interactions, all via biology-inspired approach. Antisense sequences can be immobilizd on sensor surfaces and used for DNA/RNA targets, while antibodies can be immobilized on sensor surfaces and used for detecting cytokines and other proteins. The described implementing of a multiplexing strategy to detect multiple biomarkers also strengthens the detection of a COVID-19 disease signature in patients, even in the presence of confounding biomarkers from similar diseases or disorders.

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

All embodiments of any aspect of the disclosure can be used in combination unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### EXAMPLES

Many diseases give rise to distinctive scent fingerprints, thus the balance of volatile organic compounds (VOCs) in a biological sample, e.g., human breath, can provide an indicator of health status. In the following examples, a data-driven computational method to design a multi-channel biomimetic exhaled breath VOC sensor by combining the VOC sensitivities of multiple odorant binding proteins (OBPs) and feeding their outputs into a machine learning model is provided. A combination of five OBPs (5-plex) that identify the VOC fingerprint of COVID-19 and a 10-plex that distinguishes the disease from other similar respiratory illnesses in simulation is described, as well as peptide molecular probes derived from the OBPs appended with solid-binding tags that enable the creation of sensitized carbon nanotube-based transistors to identify the VOC components of the disease. The examples further provide detail on synthesis, device binding, and signal transduction of the 5-plex solid-binding peptide probes, which were validated using VOC-spiked gas streams. All tested probes exhibited selective binding to their target analytes at approximately 1/10th of the gas concentration compared to controls, and an ethyl-butyrate-targeted carbon nanotube transistor showed a p<0.001 stronger source-drain current than a control when the gas was present. Having a means to measure VOC and optionally non-VOC content from exhaled breath can yield standard testing procedures for “breathomics”, akin to regular blood testing of cholesterol, glucose, and antibodies in determining patient health.

### Example 1: Designing an OBP Multiplex Sensor

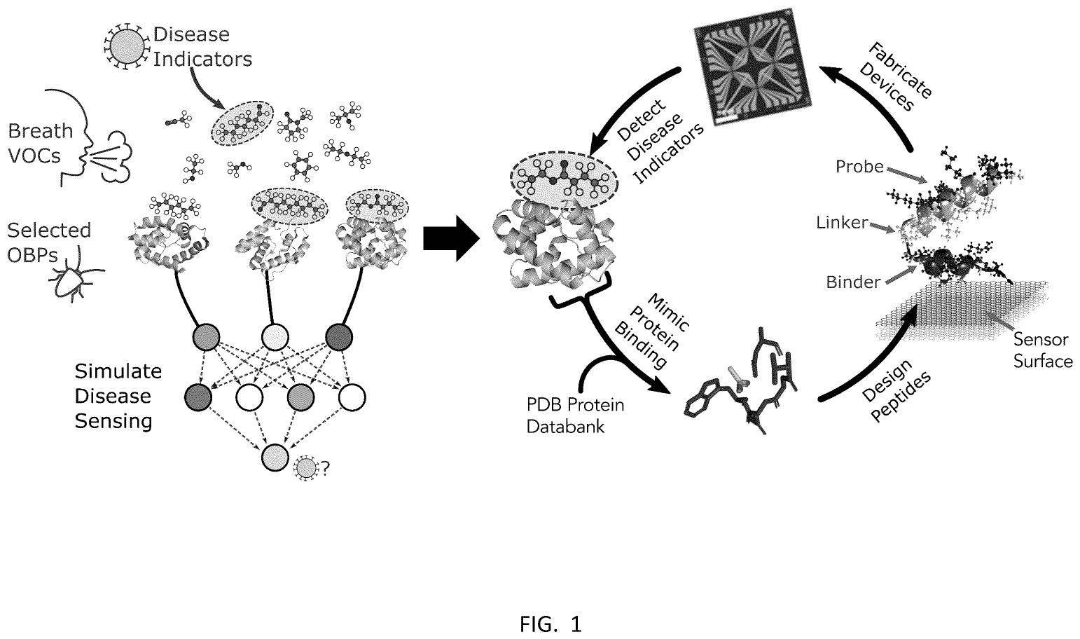

To identify disease fingerprints in human breath a machine learning (ML) classification model was developed and applied to the outputs of simulated N-plex biosensors, as illustrated in FIG. 1.

Each channel of each simulated N-plex (multiplex) biosensor utilized OBP-VOC binding affinities from one protein in the publicly available Insect Odorant Binding Protein Database (iOBPdb). The iOBPdb collates 215 independent studies of OBP-VOC interactions, comprises over 380 unique OBPs from over 90 different insect species, and specifies binding affinities to about 620 VOC targets.

Protein “sets”, N-plex in size (i.e., of various sizes), were explored by a random, combinatorial search, with the goal of identifying diseased individuals in a simulated population of breath VOC vectors, e.g., VOCs characteristic of or specific for a disease like COVID-19. One or more sets of multiplex proteins were chosen for their aggregate performance against a set of disease VOCs.

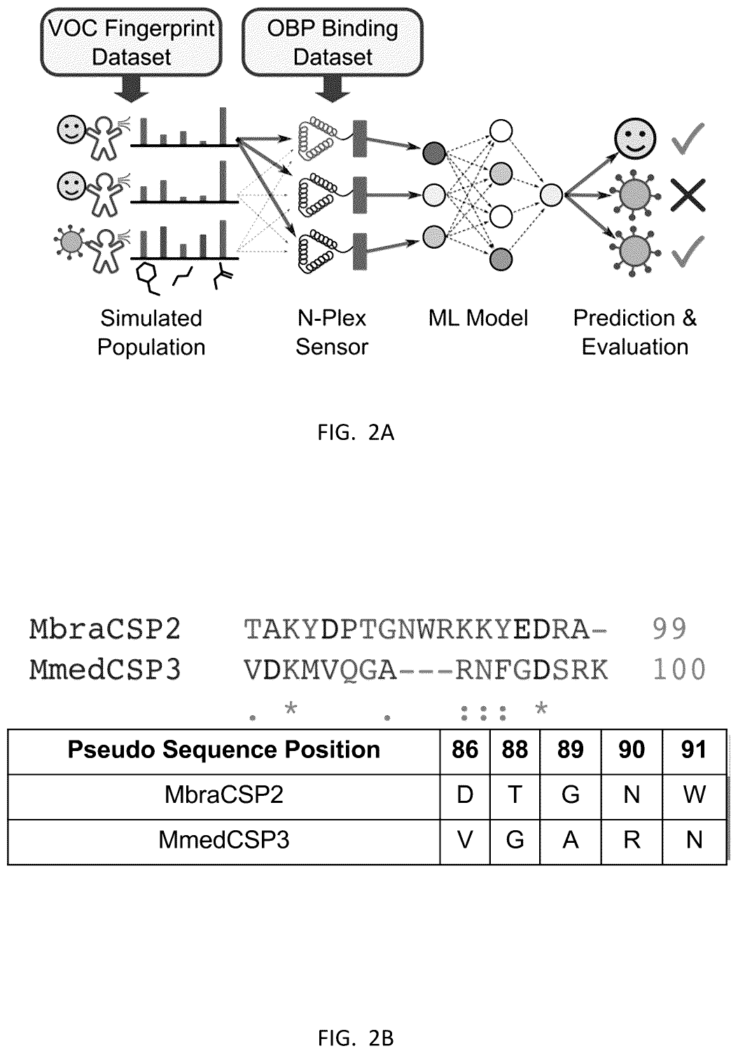

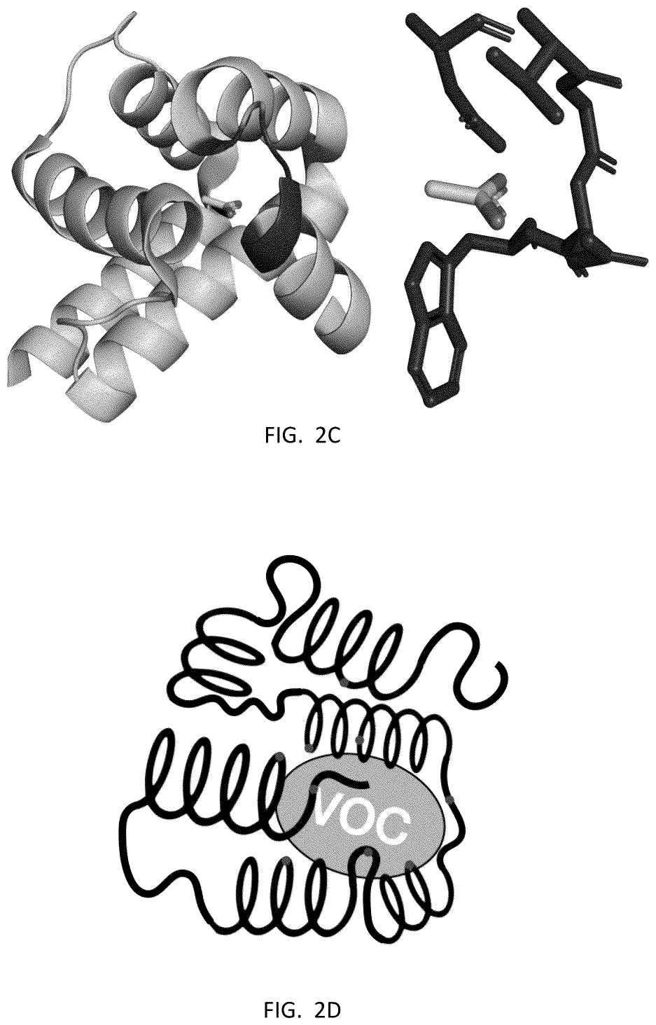

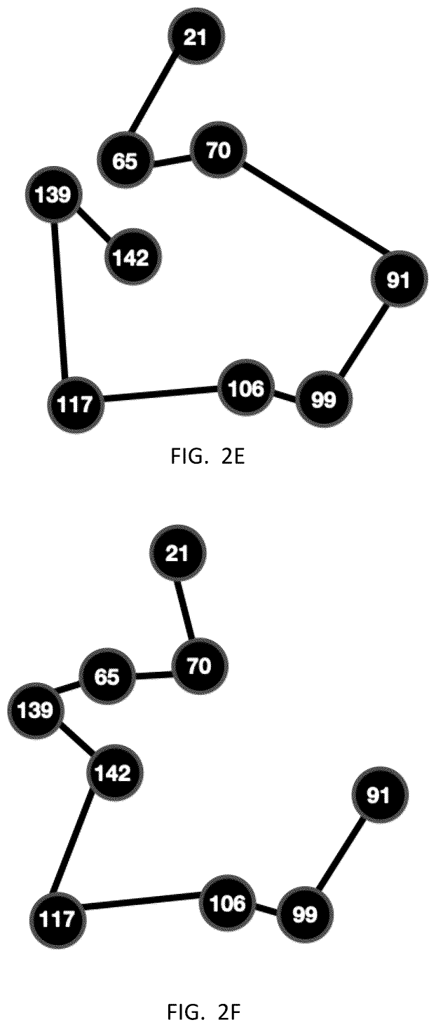

Simulated populations of VOC concentration vectors were generated by drawing independent and identically distributed random concentrations of the VOC species found in human breath (FIG. 2). Some simulated individuals showed elevated levels of the VOCs that comprise the disease fingerprint. A panel of N OBPs served as the input layer to a machine learning model, where each channel was set to a value of 1 if its associated OBP bound to any of the VOCs in the vector, or the channel would be set to a value of 0 for no binding. Alternately, the channel can estimate at what concentration factor the OBP should bind to any VOC in the vector. Both behaviors are described in greater detail below in the Methods section. The N-dimensional output from the OBP layer passed to a classifier model, which was trained to classify samples with and without the disease fingerprint. The combined OBP multiplex and model were evaluated by its “F1” score on the disease detection task (defined as the harmonic mean of the model's precision and recall).

Each OBP model was evaluated against the performance of a baseline “omniscient” model trained on the unaltered VOC vectors (i.e., without the lossy OBP layer), with the goal of identifying the smallest reasonable N where the OBP-mediated model could achieve >90% of the performance of the omniscient baseline, F1 of 0.941. In practice, N=5 sensors displayed competitive performance, with the highest-performing N=5 sensor achieving an F1 of 0.901 for COVID-19 detection, or 96% of the omniscient baseline score. This OBP 5-plex was comprised of the proteins BhorOBPm2, AfasOBP11, MmedCSP3, CpalOBP6, and HcunPBP3.

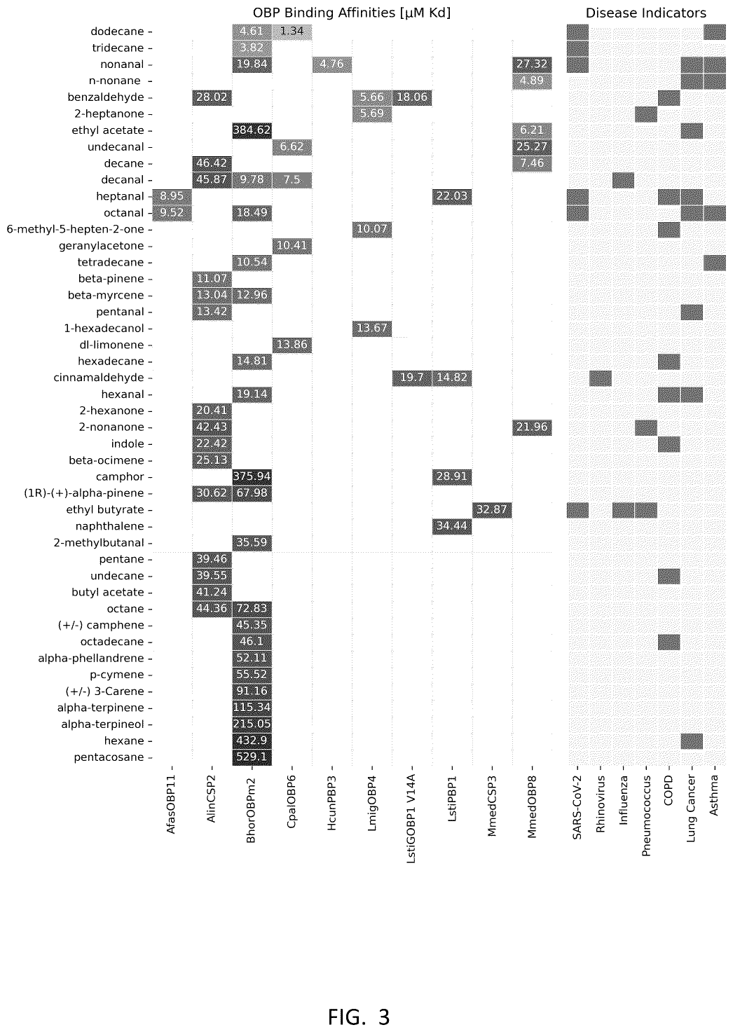

The search was repeated with individuals uniformly randomly assigned to be healthy, COVID-19 positive, or one of six other VOC signatures indicating respiratory diseases: rhinovirus, influenza, Streptococcus pneumoniae, chronic obstructive pulmonary disease (COPD), lung cancer, and asthma. With these confounding diseases present, the omniscient model achieved an F1 of 0.687. An N of 10 sensor was identified that achieved F1 of 0.622, or 91% of the baseline score. This sensor was comprised of the five COVID VOC-detecting OBPs, plus MmedOBP8, LmigOBP4, LstiPBP1, LstiGOBP1 Val14A, and AlinCSP2. The ten selected OBPs, their target VOCs, and the associated disease markers are shown in FIG. 3.

Thus, when a detection target is restricted to a single disease, we demonstrated that a massive reduction of sensor complexity (from hundreds of channels in biological systems to just five) is possible without sacrificing discriminatory performance. Ten channels were sufficient to deconvolute multiple overlapping disease signatures within the complex matrix of VOCs in human breath using machine learning models.

Further specific detail, including about breath and OBP sensor simulations, the machine learning models, and OBP optimizations, is provided in the Methods section below.

### Example 2: Probe Design by Sequence Alignment

Full length odorant binding proteins are large molecules that are relatively difficult to synthesize. Their molecular size may limit the density of possible VOC binding events on a sensor that integrates full length OBPs (e.g., carbon nanotube-based FET sensors), which would attenuate the VOC binding signal from the sensor. Peptide probe sequences were extracted from each full length OBP sequence, with the aim of identifying and isolating the VOC binding characteristics of an OBP. Extracting the VOC-binding contact residues from each OBP, as illustrated in FIG. 2, enabled integration of targeted VOC-binding probes into sensors that obviated the problems of utilizing full length OBPs. OBP VOC binding domains were combined with a sequence capable of binding to a sensor surface and, optionally, a linker, to make chimeric molecular constructs capable of being immobilized on a sensor surface and binding to a VOC.

The peptide sequence IMVTESSDYSSY is known to self-assemble on conjugated sp2 surfaces, with the YSSY motif anchoring the peptide to the substrate. This YSSY binding motif was used as the anchoring domain for chimeric molecular constructs, and a spacer was utilized to link the probe sequence and the anchoring domain. In certain experimental examples, a 3-glycene spacer domain was utilized. Thus, the amino acid sequence of peptides used to functionalize the multiplex sensor included those of the form X_{L}GGGYSSY where X_{L} is an L-length “probe” sequence that binds to a target VOC, GGG is a short linker, and YSSY is a graphite-binding motif.

For each OBP identified in a multiplex design, a binding motif was extracted from the binding slot of the protein by referencing homologically matching structures in the RCSB Protein Database (i.e., Protein Data Bank, “PDB”). A closely matching OBP structure was identified in the PDB using BLOSUM62 alignment scores, and L residues that participate in ligand binding were identified in the solved structure as described in Venthur, et al. (Physiological Entomology, 39, 183-198, 2014), in which protein structure modelling is based on the homology between a target and a template and includes four primary steps: template identification, target-template alignment, model building and model refinement and validation.

These binding slot residues were ordered using a traveling salesman search in 3D space to mimic the physical arrangement of the binding residues in the native protein structure (see FIG. 2E and FIG. 2F). Finally, the ordered list of positions was transferred back to the hero multiplex of OBPs (see OBP Multiplex Optimization in Methods section). The L amino acids at the specified positions became the OBP-derived probe X_{L}. Chimeric molecular constructs containing these probe sequences were then synthesized and their binding properties were experimentally validated.

COVID-19 VOC-targeting peptide probes are shown in Table 2. The amino acid sequence for each was extracted from the ten hero sensor OBPs by homology match, sequence alignment, and traveling salesman walk. The first five (SEQ ID NO:1 to SEQ ID NO:5) were designed to sense COVID-19 in the absence of confounding diseases and continued on to synthesis and binding affinity validation (see Table 3). The latter five (SEQ ID NO:6 to SEQ ID NO: 10) were designed to sense VOCs from the confounding diseases listed in the “Selected VOC Targets” column.

TABLE 2
OBP-derived, VOC-binding probe sequences in a 10-plex COVID-19-
detecting sensor.

| COVID-19- | COVID-19 | Matched PDB | | SEQ ID |
|---|---|---|---|---|
| specific OBPs | VOC Targets | Structure | Probe Sequence | NO |
| BhorOBPm2 | tridecane; | AgamOBP1 | GIVLLATYEKIISI | 1 |
| | dodecane; | (5EL2) | YFIFF | |
| | octanal; | | | |
| | hexanal; | | | |
| | nonanal | | | |
| HcunPBP3 | nonanal | EposPBP3 | SHFV | 2 |
| | | (6VQ5) | | |
| AfasOBP11 | heptanal; | DmelOBP28 | ITHLGVIAKNSL | 3 |
| | octanal | (6QQ4) | | |
| MmedCSP3 | ethyl butyrate | MbraCSP2 | VGARN | 4 |
| | | (1KX9) | | |
| MmedCSP3 | ethyl butyrate | MbraCSP2 | VGGGAGRGN | 33 |
| | | (1KX9) | | |
| CpalOBP6 | dodecane | DmelOBP28 | VASVVIVMLLYM | 5 |
| | | (6QQ4) | | |
| Respiratory | Selected VOC | Matched PDB | | SEQ ID |
| Disease OBPs | Targets | Structure | Probe Sequence | NO |
| MmedOBP8 | n-nonane | AgamOBP48 | KLNDARREESMG | 6 |
| | (cancer, | (4IJ7) | | |
| | asthma); ethyl | | | |
| | acetate (cancer) | | | |
| LmigOBP4 | 2-heptanone | MvicOBP3 | KRTT | 7 |
| | (strep); | (4Z39) | | |
| | benzaldehyde | | | |
| | (COPD) | | | |
| LstiPBP1 | 3-phenyl-2- | AtraPBP1 | AIVRGLSFSLM | 8 |
| | propenal | (4INW) | | |
| | (rhinovirus); | | | |
| | heptanal | | | |
| | (COVID-19, | | | |
| | COPD, cancer) | | | |
| LstiGOBP1 | benzaldehyde | BmorGOBP2 | LMSFVTFFAVME | 9 |
| V14A | (COPD); | (2WCH) | | |
| | cinnamaldehyde | | | |
| | (rhinovirus) | | | |
| AlinCSP2 | pentanal | MbraCSP2 | DTGNY | 10 |
| | (cancer); indole | (1KX9) | | |
| | (COPD) | | | |

Molecular and metadynamics can guide peptide choice, but computational modelling is also used, particularly when the little or no preexisting data on potential binding proteins exists. Methods such as AutoDock and RosettaDock can also be used, along with high-throughput signal processing methods, e.g., Resonant Recognition Model to identify the affinity of conjugate proteins binding to the same target.

### Example 3: Peptide Synthesis

Each chimeric molecular construct was synthesized using an automated solid-phase synthesizer (CS336X; CS-Bio, Menlo Park, CA, USA) through Fmoc-chemistry. In the reaction vessel, Wang resin (Novabiochem, West Chester, PA, United States), was treated with 20% piperidine in DMF to remove the preloaded Fmoc group. Incoming side chain protected amino acid was activated with hexafluorophosphate benzotriazole tetramethyl uronium (HBTU; Sigma-Aldrich, St Louis, MO, USA) in dimethylformamide (DMF, Sigma-Aldrich), incubated with the resin for 45 min, then washed with DMF. This protocol was repeated for each subsequent amino acid. The synthesis reaction was monitored by UV absorbance at 301 nm. Following synthesis, the resulting resin-bound peptides were cleaved, and the sidechain deprotected using reagent-K (TFA:thioanisole:H2O:phenol:ethanedithiol at a ratio of 87.5:5:5:2.5; Sigma-Aldrich) and precipitated by cold ether. Crude peptides were purified by RP-HPLC with >95% purity (Gemini 10 μm C18 110A column). Peptide sequences were confirmed by MALDI-TOF mass spectrometry with reflectron (RETOF-MS) on an Autoflex II (Bruker Daltonics, Billerica, MA, United States).

### Example 4: Validation of Peptide Binding to Carbon and Target VOCs

To validate peptide properties, surface plasmon resonance (SPR) was used to measure the selective binding of peptides to the carbon and quartz crystal microbalance (QCM) measurements measured selective binding of probe sequences to their target VOCs versus other profile VOCs. Exposed channels of CNT-FET devices were surface treated with the two most promising peptides, and their ability to sense a target VOC was tested in a controlled gas stream. QCM and CNT-FET device evaluations were carried out in a controlled flow N₂ environment using dry, humid, or humid VOC-spiked gas streams.

Chimeric molecular constructs described herein possess at least two binding functions: they bind selectively to a surface (e.g., to a It-conjugated carbon surface) to allow immobilization on a device, and they bind to their VOC targets. Whether a chimeric molecular construct possessed these functions was validated using SPR analysis and quartz crystal microbalance (QCM), respectively.

SPR (GE Healthcare, Biacore T200) was performed to validate specific adsorption of peptides onto a carbon substrate. Bare gold and carbon- and poly-L-lysine-functionalized substrates were used. Substrate fabrication used e-beam evaporation (CHA Industries, Solution Process Development System) of titanium (3-nm thick layer) then gold (47-nm thick layer) onto 1-cm² optical quality glass. A 20-nm thick carbon layer was sputtered (Kurt J. Lesker Company, Lab 18 Sputter System) onto the fabricated gold substrates at a rate of 10 Å per minute, and poly-L-lysine coatings were drop-cast incubated with 0.1% v/v poly-L-lysine for 30 minutes and rinsed with di-H₂O, then air dried. A single-cycle kinetics curve was generated by sequential injection of MmedPep1-GBP peptide at 2-fold increasing concentrations up to 500 nM in buffer (100 mM KCl, 10 mM K+ phosphate). An injection flow rate of 30 μL/min and a temperature of 25° C. were used. Response values were plotted and fitted to a Langmuir isotherm curve by minimizing the sum of square differences between the dataset and the isotherm model.

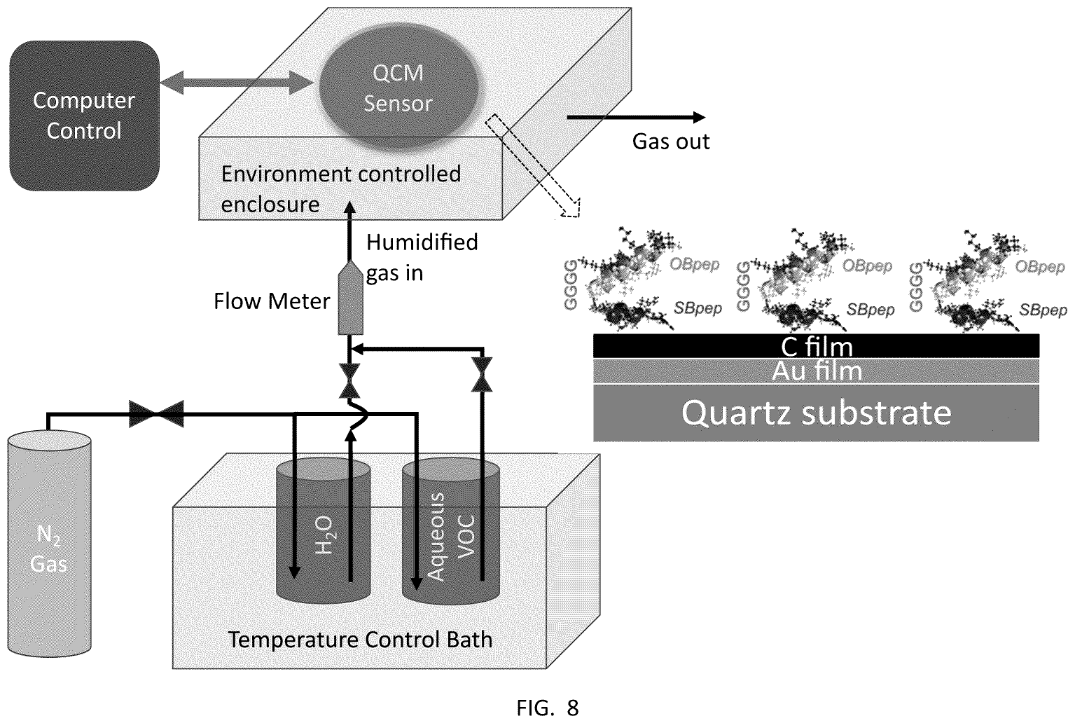

VOC binding validation by QCM utilized chimeric molecular constructs tethered to a substrate in a VOC-doped gas stream. Gold QCM substrates (Qsense, Phoenix, AZ, USA) were coated with 10 nm thick carbon film by evaporation (SPI, West Chester, PA, USA). 10 μL of 1 μM aqueous peptide was deposited and incubated for 1 hour at room temperature, and then the sample was dried in a nitrogen gas stream. A KSV model Z500 QCM (Qsense, Phoenix, AZ, USA) was used to measure VOC binding to bare carbon (control), and peptide-coated substrates. QCM measurements were performed in an air stream bubbled through a deionized water column, as shown in FIG. 8. Humidified nitrogen was measured as the baseline, then VOC was introduced by bubbling nitrogen through the VOC/deionized water mixture. The difference in resonant frequency between wet nitrogen and VOC mixture was reported, in which lower frequency indicated an increase in mass on the substrate due to VOC molecules binding to the surface.

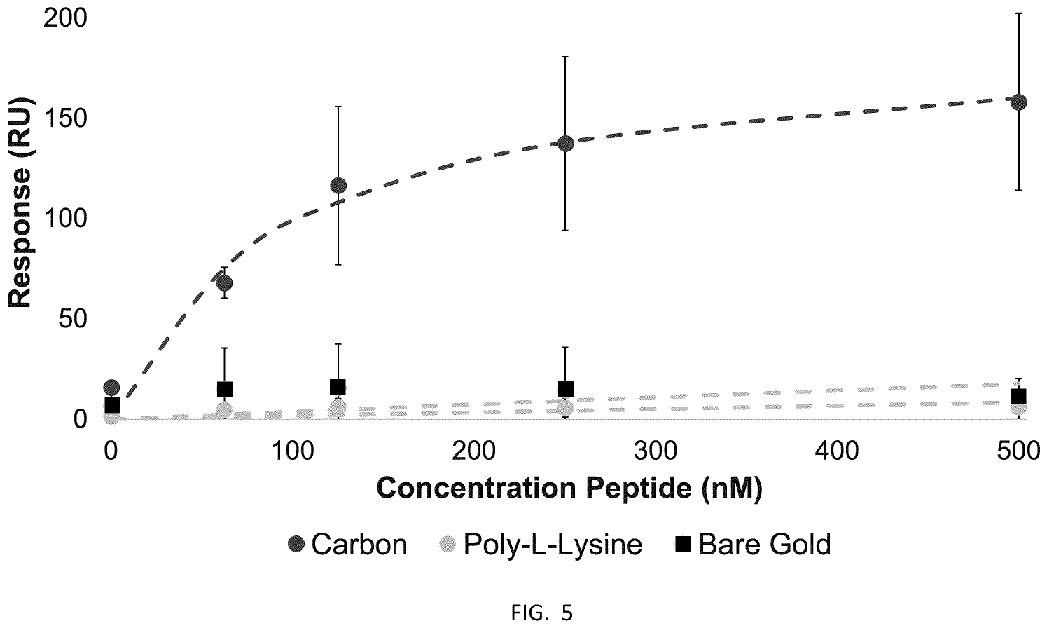

Six chimeric molecular constructs that derived from the COVID-19-sensing OBPs are shown in Error! Reference source not found. SPR results revealed a greater signal response when MmedPep1-GrBP was incubated on a carbon-coated gold surface than on either the bare gold or on poly-L-lysine. Signals of 158.4±44.1 RU were measured at 500 nM peptide concentration versus 18.4±1.7 RU and 6.5±2.9 RU for the gold and poly-L-lysine, respectively, as shown in FIG. 5. This verified that the carbon-binding sequence YSSY of MmedPep1-GrBP showed a specific preference for the carbon substrate. Table 4 describes additional chimeric molecular constructs of the disclosure.

TABLE 3
Chimeric molecular constructs synthesized for binding
affinity validation and their properties.

| | | | | SEQ | | | |
|---|---|---|---|---|---|---|---|
| Peptide | Parent | Peptide | Target | ID | MW | pI- | |
| Name | Protein | Sequence | VOCs | NO | g/mol | pH | G.R.A.V.Y. |
| MmedPep1 | MmedCSP | VGARNGGG | ethyl | 11 | 1187 | 9.58 | |
| -GrBP | 3 | YSSY | butyrate | | | | |
| MmedPep1 | MmedCSP | VGGGAGRG | ethyl | 13 | 1415 | 9.58 | −0.59 |
| s-GrBP | 3 | NGGGYSSY | butyrate | | | | |
| HcunPep1- | HcunPBP3 | SHFVGGGY | nonanal | 15 | 1160 | 7.51 | −0.22 |
| GrBP | | SSY | | | | | |
| CpalPep1- | CpalOBP6 | VASVVIVM | dodecane | 17 | 2009 | 3.51 | 1.42 |
| GrBP | | LLYMGGGY | | | | | |
| | | SSY | | | | | |
| AfasPep1- | AfasOBP1 | ITHLGVIA | octanal | 19 | 1937 | 9.56 | 0.25 |
| GrBP | 1 | KNSLGGGY | | | | | |
| | | SSY | | | | | |
| BhorPep1- | BhorOBPm | GIVLLATY | tetradecane, | 21 | 2922 | 6.68 | 1.04 |
| GrBP | 2 | EKIISIYF | tridecane, | | | | |
| | | IFFGGGYS | hexadecane, | | | | |
| | | SY | octanal, | | | | |
| | | | hexanal, | | | | |
| | | | nonanal | | | | |

TABLE 4
Additional chimeric molecular constructs

| | | | | SEQ |
|---|---|---|---|---|
| Peptide | Parent | | | ID |
| Name | Protein | Peptide Sequence | Target VOCs | NO |
| MmedPep1- | MmedCSP3 | VGARNXYSSY | ethyl butyrate | 12 |
| GrBP | | | | |
| MmedPep1s | MmedCSP3 | VGGGAGRGNXYSSY | ethyl butyrate | 14 |
| -GrBP | | | | |
| HcunPep1- | HcunPBP3 | SHFVXYSSY | nonanal | 16 |
| GrBP | | | | |
| CpalPep1- | CpalOBP6 | VASVVIVMLLYMXYSSY | dodecane | 18 |
| GrBP | | | | |
| AfasPep1- | AfasOBP11 | ITHLGVIAKNSLXYSSY | octanal | 20 |
| GrBP | | | | |
| BhorPep1- | BhorOBPm | GIVLLATYEKIISIYFI | tetradecane, | 22 |
| GrBP | 2 | FFXYSSY | tridecane, | |
| | | | hexadecane, | |
| | | | octanal, hexanal, | |
| | | | nonanal | |
| Peptide 23 | MmedOBP8 | KLNDARREESMGGGGY | n-nonane | 23 |
| | | SSY | (cancer, | |
| | | | asthma); ethyl | |
| | | | acetate (cancer) | |
| Peptide 24 | MmedOBP8 | KLNDARREESMGXYSSY | n-nonane | 24 |
| | | | (cancer, | |
| | | | asthma); ethyl | |
| | | | acetate (cancer) | |
| Peptide 25 | LmigOBP4 | KRTTGGGYSSY | 2-heptanone | 25 |
| | | | (strep); | |
| | | | benzaldehyde | |
| | | | (COPD) | |
| Peptide 26 | LmigOBP4 | KRTTXYSSY | 2-heptanone | 26 |
| | | | (strep); | |
| | | | benzaldehyde | |
| | | | (COPD) | |
| Peptide 27 | LstiPBP1 | AIVRGLSFSLMGGGYS | 3-phenyl-2- | 27 |
| | | SY | propenal | |
| | | | (rhinovirus); | |
| | | | heptanal | |
| | | | (COVID-19, | |
| | | | COPD, cancer) | |
| Peptide 28 | LstiPBP1 | AIVRGLSFSLMXYSSY | 3-phenyl-2- | 28 |
| | | | propenal | |
| | | | (rhinovirus); | |
| | | | heptanal | |
| | | | (COVID-19, | |
| | | | COPD, cancer) | |
| Peptide 29 | LstiGOBP1 | LMSFVTFFAVMEGGGY | benzaldehyde | 29 |
| | V14A | SSY | (COPD); | |
| | | | cennamaldehyde | |
| | | | (rhinovirus) | |
| Peptide 30 | LstiGOBP1 | LMSFVTFFAVMEXYS | benzaldehyde | 30 |
| | V14A | SY | (COPD); | |
| | | | cennamaldehyde | |
| | | | (rhinovirus) | |
| Peptide 31 | AlinCSP2 | DTGNYGGGYSSY | pentanal | 31 |
| | | | (cancer); indole | |
| | | | (COPD) | |
| Peptide 32 | AlinCSP2 | DTGNYXYSSY | pentanal | 32 |
| | | | (cancer); indole | |
| | | | (COPD) | |

Where present, “X” is an amino acid linker

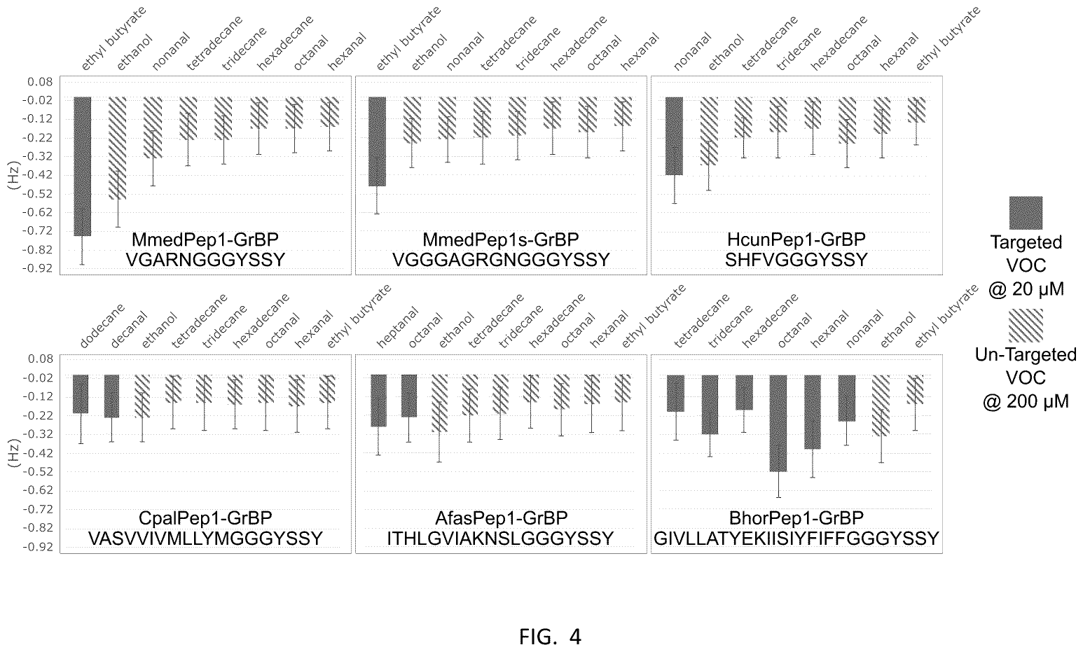

Each of the six synthesized peptides was then tested against eight VOCs. The VOCs were chosen to include the targeted VOCs for all synthesized chimeric molecular constructs, plus ethanol as a common non-target. FIG. 4 shows the QCM frequency change from wet N₂ gas to VOC-bearing wet N₂ gas. VOC adsorption caused damping of the QCM sensor—therefore, a higher affinity surface would produce a greater reduction in resonant frequency upon exposure to the gas stream. The concentration of the target VOC for each peptide was 20 μM, 10× lower than non-targets at 200 μM. All peptides tested exhibited greater binding to their targets even at 10× lower concentration, excluding ethanol, indicating differentiation between targeted and non-targeted VOCs. However, HcunPep1-GrBP's nonanal affinity and BhorPep1-GrBP's octanal affinity were notable and the MmedOBP8-derived peptides showed a marked preference for ethyl butyrate binding, just like their parent OBPs.

The peptide variant MmedPep1s-GrBP was intended to evaluate the incorporation of glycine residues in its “probe” block to replicate the physical distances between residues in the protein's native binding slot. This spacer variant was also strongly selective for ethyl butyrate, with reduced affinity for ethanol.

### Example 5: CNT Device Fabrication

Carbon nanotube field-effect transistor (CNT FET) devices were fabricated that enable peptide-sensitized detection of VOC targets from the vapor phase in a bottom gate configuration. Semiconducting single wall carbon nanotubes (SWCNT; from NanoIntegris) were chosen for a high on/off ratio and a low noise floor near and below the threshold voltage. The devices featured four spatially resolved sensing regions, each with eight transistors for 32 total sensors on each chip. Each region had a common source electrode and could be functionalized with a different VOC-binding chimeric molecular construct.

A photoresist-free, solution-deposition fabrication was used to minimize device hysteresis, which produced devices with transfer curve on-off ratios >10³ and threshold voltages <5 V, as measured by a Keithley 2450 source meter. In brief, a back gate was made by reactive ion etching with CF₄/CHF₃ to remove the silicon dioxide layer on the back side of the silicon wafer, followed by deposition of chromium and gold. For the electrode contacts, photolithography then metallization and liftoff procedures were carried out. AZ1512 photoresist was then used to create channel banks in the sensing regions, in which CNT solution was dropcast incubated prior to functionalization with chimeric molecular construct.

Specifically, the quadruplex multiarray sensor was fabricated as follows. Photolithography was performed on a silicon wafer (WaferPro cat. X040061000W, Santa Clara, CA, USA) to create the source and drain elements and electrical contact pads. NR9-3000PY negative tone photoresist was first spin coated on the wafer, then aligned with a photomask bearing the pattern for the source, drain, and contact pad components. Exposure to a 180 mJ UV dose was then performed, followed by development in AD10 solution to reveal the photolithographic pattern. Metallization of electrical components was carried out by sputtering a 9-nm chromium bonding layer and 70-nm gold layer.

A back gate was also created on the underside of the wafer by reactive ion etching with CF₄/CHF₃ gas to remove the silicon dioxide layer, followed by deposition of 21 nm Cr and 105 nm Au. Metal lift-off was accomplished by incubating in acetone for 1.5 hours, then in isopropanol with sonication for 1-3 minutes. Channel banks were also created that would accommodate drop casting of the CNT solution into the sensing channels via photolithography with AZ1512 positive tone photoresist and AZ340 developer solution. Devices were cleaned with O₂ plasma for 1-3 minutes.

Aqueous semiconducting CNT solution (NanoIntegris; IsoNanotubes-S; Boisbriand, QC, Canada) was dispensed into the channel banks for a photoresist-free process intended to minimize IV curve hysteresis that would eliminate any potential photoresist residue contamination on the CNT layer. CNT solution was incubated on the device for 60 to 120 minutes, followed by gentle washing with deionized H₂O (FIG. 6A, FIG. 6B). Sensing channel dimensions were either 200 μmL×50 μm W or 25 μmL×100 μm W. For signal measurement data, responses were normalized to their respective W:L ratios.

### Example 6: CNT FET Device Characterization

Fabricated e characterized by assessing current-voltage characteristics. CNT FET sensors were connected to two Keithley 2450 source measure units (SMU; Tektronix, Beaverton, OR, USA) in a 2-wire configuration, whereby one SMU applied the source-drain voltage potential and the other SMU applied the source-gate potential. XYZ-axis micromanipulators were used to position tungsten probe tips (Form Factor; No. 107-157, 1.5 μm; Livermore, CA, USA) onto the contact pads of source and drain electrodes. For tuning the back gate, copper tape was affixed to the gold-deposited underside of the sensor device and clamped with an alligator clip connected to the gate SMU. Transfer curves were obtained by forward sweeping the gate voltage potential from 0 V to +3 V followed by reverse sweeping to −3 V and back to 0 V, while stepping the source-drain potential from −1 V to +5 V with +2-volt step sizes. Alternatively, transfer curves were also taken by forward sweeping the gate potential from −5 V to +5 V and then reverse sweeping back down to −5 V.

Charge carrier mobility (u) was calculated using the following formula:

$$μ = ( \frac{{dI}_{ds}}{d {V}_{g}} ) ( \frac{L}{W \cdot {C}_{ox} \cdot {V}_{ds}} )$$

where I_{ds} is the current across the source and drain, V_{g} is the gate voltage, L and W indicate FET channel dimensions, and V_{ds} is the source-drain voltage. The capacitance of the silicon dioxide layer, Cₒₓ, is defined as:

$${C}_{o x} = \frac{ε \cdot {ε}_{r}}{d}$$

in which ε represents the vacuum permittivity constant 8.854×10⁻¹² F/m, and εᵣ is the relative permittivity of silicon dioxide with a value of 3.9. The thickness of the SiO₂ dielectric layer, d, is 100 nm. Mean mobility and standard error values were obtained from 32 CNT FET sensors split between two chips.

Threshold voltages were determined from the transfer curves by taking the x-intercept from the linear portion of the reverse sweep. Mean values along with standard deviation were calculated from 20 sensors from the chip bearing channel dimensions of 200 μmL×50 μm W.

On-off ratios were obtained from the transfer curves with the larger sweep range for the gate voltage (i.e., −5 V to +5 V) and were calculated by dividing the maximum source-drain current by the minimum source-drain current (n=31 from two chips). Results were reported as mean values with standard deviation.

### Example 7: VOC Gas Stream Experiments and Peptide Functionalization of CNT FET Sensors

Sensors were placed in a box chamber that allowed gas flow into and out of the enclosure while allowing micromanipulator probes to be positioned on the sensor chip contact pads. Gas flow impinged directly above the sensor chip with a continuous flow rate of 50-60 SCCM as controlled by a flowmeter (Matheson, model FM-1050, Montgomeryville, PA, USA).

After placing the sensor chip into the gas chamber, a nitrogen purge of 2-3 minutes at 4 to 5 PSI in the gas line was performed in order to flush out ambient air trapped within the box enclosure. Transfer curve measurements were performed to determine sensor characteristics. The chip was then equilibrated in humidified N₂ (“wet N₂”) for 30-60 min by flowing in nitrogen gas that has passed through a gas bubbler filled with water. Sensor measurements were taken at this point to establish sensor baseline signal.

For peptide sensitization of the CNT sensing channel, the chip was removed from the gas chamber and dropcasted with 1.5 μL 100 μM peptide diluted in di-H₂O. Peptide solution was pipetted directly onto the CNT sensing regions of the multiplexed chip and incubated for 60 minutes in a humidified environment to prevent evaporation. Devices were then spin dried by centrifugal force at 2,000 g. Sensor measurements were taken again after replacing the chip back into the gas chamber, purging with N₂, and equilibrating in wet N₂ for 30-60 min. Finally, the peptide-functionalized chip was exposed to humidified ethyl butyrate for 60-90 min before sensor measurements were taken.

### Signal Response Analysis

Transfer curves were generated for each of the following experimental conditions: 1) Bare CNT sensors (i.e., non-functionalized) under non-humidified N₂ gas exposure; 2) non-functionalized sensors under humidified N₂ gas exposure; 3) peptide-functionalized sensors under humidified N₂ gas exposure; and 4) peptide-functionalized sensors exposed to ethyl butyrate in humidified N₂ carrier gas. Signal response was based on normalized source-drain current values obtained from the reverse sweep of the transfer curve at a gate potential of −1 V (V_{g}=−1 V), which was determined to be within the subthreshold voltage region for ethyl butyrate-exposed, peptide-functionalized sensors. Source-drain potential was −5V (V_{sd}=−5 V), which gave a more robust signal response compared to other source drain potentials tested. Signal responses were normalized to their corresponding width-to-length CNT sensing channel dimensions. Mean signal response for each condition was calculated from a total of 9 to 13 technical replicates measured across two independent experiments (N=2).

Transfer curves revealed low threshold voltage, high dynamic range FETs, allowing for low voltage operation (FIG. 6C). Device characteristics under dry nitrogen included a threshold voltage of −1.32±0.77 V and device on-off ratios on the order of 10³ (1247±1556 V). Charge mobilities were calculated to be 1.34±0.92 cm² V⁻¹ s⁻¹ at a gate voltage of −5 V, which is consistent with other reported organic and carbon-based back gated transistors that range from 0.37 to 30 cm² V⁻¹ s⁻¹.

### Example 8: VOC Detection by Peptide-Sensitized Carbon Nanotube Transistors

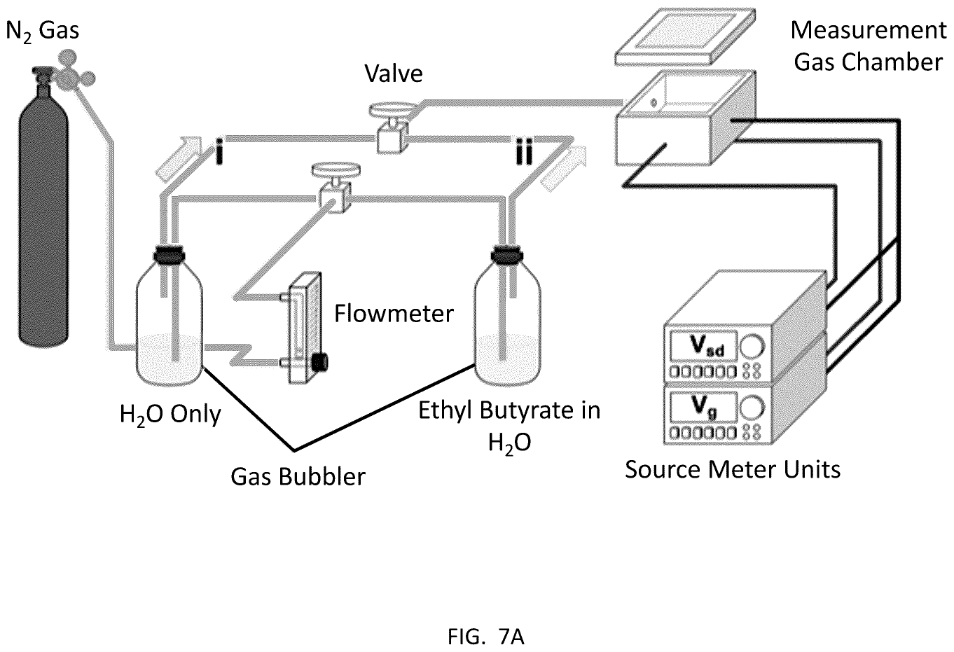

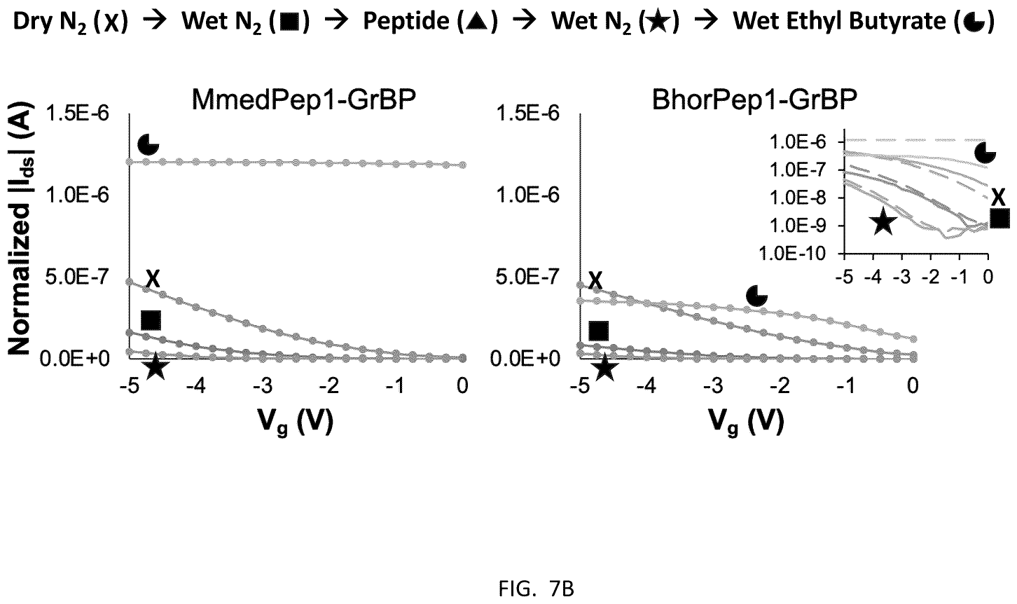

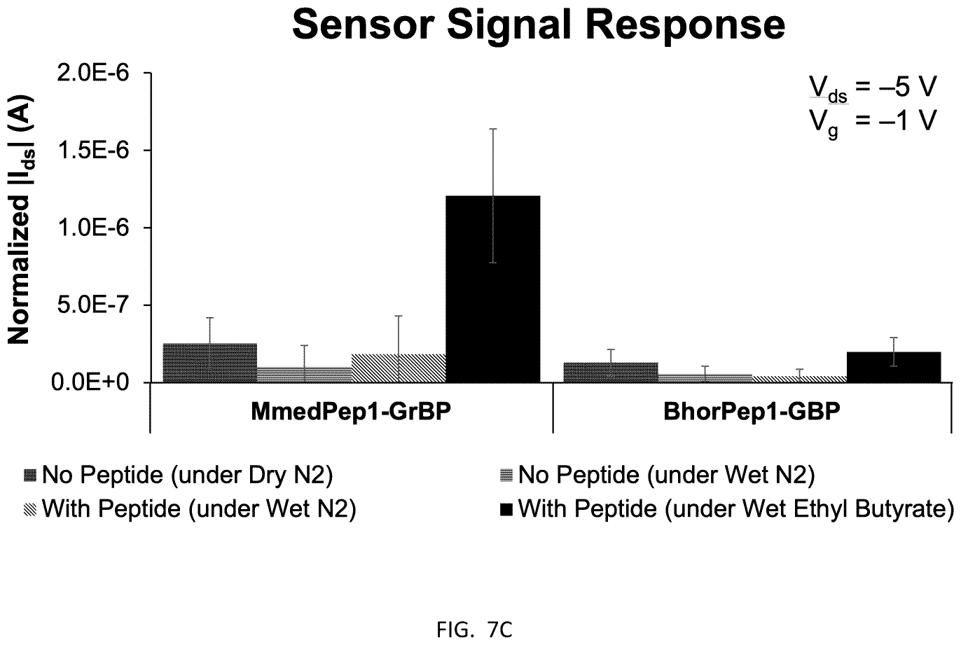

Two VOC and C-binding peptides were used in order to functionalize CNT-FET devices and test their sensitivity to ethyl butyrate:MmedPep1-GrBP and BhorPep1-GrBP, which were predicted to bind to ethyl butyrate and a range of aldehydes and alkanes, respectively. Low source-drain currents were observed for bare CNT devices without peptide or for peptide-sensitized devices in an N₂ gas stream when monitoring the sensing channel at a back gate voltage of −1 V for a 100 nm SiO2 gate dielectric. Bare CNT responses showed a successive decrease in current when exposed to humidified nitrogen and further decreased after peptide functionalization. However, the effect was reversed under ethyl butyrate exposure for CNT channels functionalized with MmedPep1-GrBP (FIG. 7B). Specifically, upon exposure to 378 mM ethyl butyrate solution (5% v/v in H₂O), MmedPep1-GrBP-sensitized CNT-FET sensors measured 1.21±0.43 μA of current, compared to 0.20±0.9 μA for the aldehyde-sensitive BhorPep1-GrBP (p<0.001), as shown in FIG. 7C. This indicates that (i) the selective binding observed in QCM translated to signal transduction in a CNT-FET device, and (ii) the response was specific, contingent on the binding tendencies of the peptides on each channel.

Signal responses were also taken at other source-drain potentials. At V_{ds}=−3 V, the CNTs sensitized with MmedPep1-GrBP showed a current of 0.542±0.721 μA (at a gate voltage of −1 V) upon ethyl butyrate exposure, while BhorPep1-GrBP-sensitized CNTs exhibited 0.038±0.020 μA. The signal difference between these two peptides was statistically significant (p<0.05). However, at V_{ds}=−1 V, which is closer to the threshold region of the transistor response, the difference in signal response between the two peptides was minimal with currents of 0.012±0.0.95 μA and 0.019±0.0.47 μA for MmedPep1-GrBP and BhorPep1-GrBP, respectively. This discrepancy provides justification for using higher source-drain potentials, further from the threshold region, to elicit a more robust sensor response.

### Methods

### Designing an OBP Multiplex Sensor: Simulated Populations of VOCs

One simulated breath was generated by drawing a concentration value c for each common breath VOC from a log-normal distribution: c∈exp( [symbol] (μ, σ)). A “sick” individual was simulated by increasing the mean of the distribution of the VOCs in the COVID-19 fingerprint by a scaling factor of v, i.e., c_{COVID}∈exp( [symbol] (μ+v, σ). Larger v effectively reduced the difficulty of the diagnosis task by boosting the disease fingerprint. In practice, v was tuned to produce a realistic result in a baseline model. Each synthetic population comprised M vectors C={c₁, c₂, . . . c_{V}} of V VOC concentrations, with some fraction possessing elevated concentrations from the disease fingerprint. To design a multiplex sensor, the ability of a set of OBPs to bind VOCs, in combination with a model to classify diseased and non-diseased vectors in the synthetic population, was optimized in a virtual testbed.

### Designing an OBP Multiplex Sensor: Simulated OBP Sensors

Each unique OBP specifically binds to a unique subset of VOCs. To model this behavior, the testbed deployed one of two rules for OBP-VOC signal transduction. The first rule assumed nonlinear OBP transduction, that they activate in a binary manner and do not distinguish between binding events to different VOCs. An OBP with experimentally determined binding affinities

$${A}_{P} = \{ {a}_{P}^{c 1} , {a}_{P}^{c 2} , … \}$$

would “activate” when any C>A_{P}, that is

$$p ( C ) = {V}_{i = 1}^{V} ( {c}_{i} > {a}_{P}^{c i} ) .$$

The second rule assumed that a gas mixture could be scaled linearly over time in a device, enabling reporting of a “time” scaling factor accounting for the time at which an OBP activated. That is,

$$p ( C ) = arg {min}_{t} ( {⋁}_{i = 1}^{V} ( {tC}_{i} > {a}_{P}^{c i} ) = True ) .$$

Both rules were conservative, assuming no linear response to VOC concentrations and an inability of any single OBP to differentiate between different bound VOC species. The 5-plex COVID-19 OBPs were identified using the binary VOC sensor model, and the 10-plex multi-disease sensor was identified using the time-scaling VOC model.

The first layer of each prospective disease classification model consisted of a set of N OBP proteins, each of which read the input VOC vector according to the binary or time-scaling activation function p. An N-plex OBP sensor measured the VOC vectors C from the population to produce the N-long sensor readings P_{OBP}=[p₁(C), p₂(C), . . . p_{N}(C)]. The N-plex was then be evaluated based on the ability of a model to classify diseased individuals using inputs P_{OBP}.

### Designing an OBP Multiplex Sensor: Machine Learning Classification

The N-dimensional vector created by the simulated OBP N-plex was passed as input to a classification machine learning model. All models were implemented using adaptive boosted decision tree ensembles using Python programming language machine learning toolkits from SciKitLearn, AdaBoostClassifier with DecisionTreeClassifier base estimators. Each model f accepts an input vector of VOC concentrations or OBP responses to VOC concentrations, generated as described above. It produces y′, the predicted binary disease state of the vector.

### Designing an OBP Multiplex Sensor: Model Evaluation

Each multiplex sensor set X was evaluated on its ability to accurately train a machine learning model to classify healthy and diseased individuals in a virtual testbed. A mixed population of M VOC vectors was drawn 50%/50% from healthy and diseased distributions, and the population was split randomly into 90%/10% train and test sets.

An “omniscient” classifier model was evaluated as a baseline. This model received an input of the true VOC concentrations C from the synthetic population, i.e., fₒₘ(C)→y′. Model fₒₘ trained on the training set and reported an F1 score, defined as F1=2 (precision·recall)/(precision+recall), on the task of identifying VOC vectors drawn from the diseased distribution in the test set. The performance of the omniscient model was retained as a comparison point for all subsequent models.

OBP multiplex sets were then evaluated on the same task and VOC population using the interface f(P_{X}(C))→y′, where P_{X} is the OBP multiplex sensing operation using candidate OBP set X as its input layer and f is the random forest model trained on the OBP multiplex's outputs. For each proposed OBP set X, the F1 score of the combined model f(P_{X}(C)) was produced.

### Designing an OBP Multiplex Sensor: OBP Multiplex Optimization

To select a hero multiplex of OBPs, channels (i.e., OBP proteins) were selected one at a time to account for conditional relationships between OBPs. For instance, a nonanal-binding OBP may have high utility for detecting a disease but adding a second nonanaldehyde binder might be expected to provide minimal incremental benefit. Therefore, each OBP was evaluated in the context of the other specific OBPs in an N-plex set. An N-plex was thereby built piecewise, so that sensor tests were conditioned on all preceding OBP choices. The operation was defined as follows:

Begin with the “hero sensor” and “best-yet sensor” comprising empty sets H=Ø and B^{†}=Ø and T total experiments budgeted. Define the function E(X) to be the evaluation operation described above that returns an F1 score for a sensor comprised of OBPs X:
Randomly select T/N prospective sets of N−len(H) OBPs without replacement—this set of OBP sets is S;
Evaluate each prospective set, retaining the highest performer: B′=argmax_{BES}E(H∪B);
If B′ had a higher F1 score than B^{\}, B^{†}←B′;
Select the OBP o′ that most reduces the sensor's effectiveness when removed:

$${o}^{′} = arg {min}_{o ∈ {B}^{†}} E ( H ⋃ {B}^{†} - o ) ;$$

Append the new OBP to the hero sensor: H←H∪o′ and B^{†}←B^{†}−o′
Repeat steps 1-5 until len(H)=N

The intended output from this process was a set of N OBPs that maximize the F1 score on the classification task of identifying a disease by its VOC fingerprint. Optimization was performed on the Hyak supercomputer at the University of Washington, allowing about 100,000 potential multiplex sets to be evaluated per day on the disease classification task. In total, less than 1 million multiplex sets were evaluated to generate the specific sets presented herein.

### Designing an OBP Multiplex Sensor: Hyperparameter Optimization

All random forest and decision tree models and training behaviors used default hyperparameters, with the exception of the decision tree max depth, which was set to 1. No hyperparameter optimization was performed before or during model evaluation.

Scaling values for μ, the VOC population average, and v, the disease scaling value, were chosen because present studies of COVID-19 VOCs are directional rather than quantitative—it was unknown to what degree VOC concentrations are perturbed in sick individuals. Qualitatively, perturbations between 2× and 10× appear likely. This parameter choice was potentially of significant import as an unsuitably small v renders the detection task impossible, while an unsuitably large choice makes it trivial. Canine diagnostic studies have demonstrated diagnostic sensitivity in the 95% confidence interval of 93.1-97.6% and specificity within a 95% confidence interval of 90.7%-100.0%. Generously assuming omniscient VOC concentration detection for dogs, we and chose u and v so that the omniscient baseline sensor would mimic the canine performance, achieving an F1 score in the range 0.93 to 0.97.

All experiments used M=5,000 simulated VOC populations, chosen to represent the upper end of realistic sample size in an early-phase human trial campaign. With this sample size, manual experimentation found that the naive choice of μ=v=7, for a 2× increase in disease VOC levels, yielded an omniscient classifier 0.941 F1 score, which was within the target performance range above. The performance of the omniscient model then served as the baseline for evaluating sets of OBPs.

The falloff when introducing confounding disease signatures was notable, to an F1 of about 0.6. The choice not to re-tune v allowed for direct comparison between the two studies and emphasized the challenge of this task with confounding diseases present. Because model performance was so sensitive to the choice of v, conclusions were drawn only from the relative performance of sensors to the omniscient baseline.

1-2. (canceled)

3. A chimeric molecular construct comprising one or more peptides comprising an amino acid sequence at least 90%, 95%, or 100% identical to the amino acid sequence selected from SEQ ID NOS: 1-10 or SEQ ID NO: 33 fused to one or more functional domain and one or more linker sequence linking the peptide and the functional domain.

4. The chimeric molecular construct of claim 3, wherein the one or more functional domain comprises a surface binding domain.

5. The chimeric molecular construct of claim 4, wherein the surface binding domain comprises or consists of an amino acid sequence at least 90%, 95%, or 100% identical to the amino acid sequence selected from the group consisting of SEQ ID NOS: 34-64.

6. The chimeric molecular construct of claim 5, wherein the surface binding domain comprises an amino acid sequence at least 75%, or 100% identical to the amino acid sequence YSSY.

7. The chimeric molecular construct of claim 3, comprising an amino acid sequence at least 90%, 95%, or 100% identical to the amino acid sequence selected from the group consisting of SEQ ID NOS: 11-32.

8. A composition comprising 1, 2, 3, 4, 5, 6, 7, 8, 9, 10, or more of the chimeric molecular constructs of claim 3.

9. The composition of claim 8, comprising 1, 2, 3, 4, 5, or all 6 of the following:
(a) at least one amino acid sequence at least 90%, 95%, or 100% identical to the amino acid sequence of SEQ ID NOS: 11-12;
(b) at least one amino acid sequence at least 90%, 95%, or 100% identical to the amino acid sequence of SEQ ID NOS: 13-14;
(c) at least one amino acid sequence at least 90%, 95%, or 100% identical to the amino acid sequence of SEQ ID NOS: 15-16;
(d) at least one amino acid sequence at least 90%, 95%, or 100% identical to the amino acid sequence of SEQ ID NOS: 17-18;
(e) at least one amino acid sequence at least 90%, 95%, or 100% identical to the amino acid sequence of SEQ ID NOS: 19-20; and
(f) at least one amino acid sequence at least 90%, 95%, or 100% identical to the amino acid sequence of SEQ ID NOS: 21-22.

10. The composition of claim 9, further comprising 1, 2, 3, 4, or all 5 of the following:
(a) at least one amino acid sequence at least 90%, 95%, or 100% identical to the amino acid sequence of SEQ ID NOS: 23-24;
(b) at least one amino acid sequence at least 90%, 95%, or 100% identical to the amino acid sequence of SEQ ID NOS: 25-26;
(c) at least one amino acid sequence at least 90%, 95%, or 100% identical to the amino acid sequence of SEQ ID NOS: 27-28;
(d) at least one amino acid sequence at least 90%, 95%, or 100% identical to the amino acid sequence of SEQ ID NOS: 29-30; and
(e) at least one amino acid sequence at least 90%, 95%, or 100% identical to the amino acid sequence of SEQ ID NOS: 31-32.

11. The composition of claim 8, wherein the 1, 2, 3, 4, 5, 6, 7, 8, 9, 10, or more of the chimeric molecular constructs are bound to a surface.

12. The composition of claim 11, wherein the surface comprises a carbonaceous surface, and wherein the 1, 2, 3, 4, 5, 6, 7, 8, 9, 10, or more of the chimeric molecular construct are non-covalently bound to the carbonaceous surface.

13. The composition of claim 12, wherein the surface comprises a carbon nanotube, including but not limited to a single wall carbon nanotube or multiwall carbon nanotube.

14. The composition of claim 12, wherein the surface comprises a carbon nanotube field-effect transistors (CNT-FET), or a graphene field effect transistor (gFET)

15. The composition of claim 11, wherein the surface comprises a one dimensional semiconducting element, a two dimensional semiconducting element, an oxide, II-VI, III-V or group IV bulk or thin film semiconductor, a semiconductor or semimetal, or a dielectric or protective layer.

16. The composition of claim 11, wherein the composition comprises a plurality of transistors, and wherein a single peptide or chimeric molecular construct is immobilized on a surface of each transistor in the plurality of transistors.

17. The composition of claim 16, wherein each transistor comprises a different peptide or chimeric molecular construct.

18. A method for detecting, prognosing, or monitoring treatment for COVID-19 infection, comprising
(a) contacting the composition of claim 12 with a biological sample;
(b) detecting volatile organic compounds (VOCs) in the biological sample by their binding to the peptides or chimeric molecular constructs present in the composition; and
(c) comparing a signature of VOCs present in the biological sample with a VOC signature characteristic of COVID-19; wherein a VOC signature present in the sample that matches a VOC signature characteristic of COVID-19 serves to detect COVID-19 infection, prognosis, or response to treatment in the biological sample.

19. The method of claim 18, wherein the VOC signature present in the sample is converted to an electrical, photonic, magnetic, acoustic, colorimetric, optical, or other metric signal for the comparing step.

20. The method of claim 18, wherein the comparing steps comprise use of a computational model trained to differentiate biological samples with and without the VOC signature characteristic of COVID-19.

21. (canceled)

22. The chimeric molecular construct of claim 3, wherein the linker is absent.

23-81. (canceled)

82. The chimeric molecular construct of claim 3, wherein the one or more peptides comprise an amino acid sequence at least 90%, 95%, or 100% identical to the amino acid sequence selected from SEQ ID NOS: 11-22, fused to one or more functional domain and one or more linker sequence linking the peptide and the functional domain.
